(19) United States
(12) Patent Application Publication
(10) Pub. No.: US 2025/0051731 A1
(43) Pub. Date: Feb. 13, 2025

(54) COMPOSITIONS AND METHODS FOR ESTABLISHMENT OF BOVINE-INDUCED PLURIPOTENT STEM CELLS

(71) Applicant: University of Connecticut, Farmington, CT (US)

(72) Inventors: Young Tang, Farmington, CT (US); Yue Su, Farmington, CT (US); Ling Wang, Farmington, CT (US); Xiuchyn Tian, Farmington, CT (US); Jiaqi Zhu, Farmington, CT (US)

(21) Appl. No.: 18/721,402

(22) PCT Filed: Dec. 20, 2022
(86) PCT No.: PCT/US2022/081995

§ 371 Date: Jun. 18, 2024

Related U.S. Application Data
(60) Provisional application No. 63/291,669, filed on Dec. 20, 2021.

(51) Int. Cl. C12N 5/074

(52) U.S. Cl. CPC C12N 5/0696; C12N 2501/235; C12N 2501/415; C12N 2501/602; C12N 2501/603; C12N 2501/604; C12N 2501/605; C12N 2501/606; C12N 2501/608; C12N 2501/71; C12N 2501/72; C12N 2501/727; C12N 2506/1346

(57) ABSTRACT

The disclosure provides bovine-induced pluripotent stem cells along with compositions and methods for use in producing the same.

### STATEMENT REGARDING RELATED APPLICATIONS

This application claims priority to U.S. Provisional Application No. 63/291,669, filed Dec. 20, 2021, the entire contents of which are incorporated herein by reference for all purposes.

### STATEMENT REGARDING FEDERAL FUNDING

This invention was made with government support under Grant Nos. 2016-67016-24894 and 2019-67015-29413 awarded by the United States Department of Agriculture. The government has certain rights in the invention.

### SEQUENCE LISTING

The text of the computer readable sequence listing filed herewith, titled “40105_601_SequenceListing”, created Dec. 14, 2022, having a file size of 11,662 bytes, is hereby incorporated by reference in its entirety.

### FIELD

The present disclosure relates to bovine-induced pluripotent stem cells along with compositions and methods for use in producing the same.

### BACKGROUND

There have been numerous efforts in the derivation of bovine pluripotent stem cells (PSCs) including bovine embryonic stem cells (bESCs) and induced pluripotent stem cells (biPSCs). Attempts to derive bESCs from the inner cell mass (ICM) of bovine embryos started more than 22 years ago [4]. However, the establishment of biPSCs has proven challenging, due to issues including differentiation within a few passages, limited self-renewal capacity, and/or the requirement for exogenous transgenes for contiguous propagation. Accordingly, there is a long unmet need for methods for producing bovine-induced iPSCs.

### SUMMARY

In some aspects, provided herein are compositions. In some embodiments, the compositions are cell culture media.

In some embodiments, provided herein is a cell culture medium comprising a WNT inhibitor and a histone methyltransferase DOT1L inhibitor. In some embodiments, the WNT inhibitor comprises IWR1 and wherein the DOT1L inhibitor comprises EPZ004777. In some embodiments, the cell culture medium further comprises human leukemia inhibitory factor (LIF).

In some embodiments, provided herein is a cell culture medium comprising a WNT inhibitor, a histone methyltransferase DOT1L inhibitor, human leukemia inhibitory factor (LIF), and an adenyl cyclase activator. In some embodiments, the adenyl cyclase activator is forskolin. In some embodiments, the cell culture medium further comprises at least one glycogen synthase kinase 3 (GSK-3) inhibitor and at least one mitogen-activated protein kinase inhibitor.

In some aspects, provided herein are methods of producing cells. In some embodiments, provided herein is a method of producing a bovine-induced pluripotent stem cell. In some embodiments, the method comprises providing a bovine mesenchymal stem cell, and inducing overexpression of a plurality of reprogramming factors in the bovine mesenchymal stem cell. In some embodiments, the plurality of reprogramming factors comprise lysine-specific demethylase 4A (KDM4A), OCT4, SOX2, KLF4, cMYC, LIN28, and NANOG. In some embodiments, the method further comprises culturing the mesenchymal stem cells under suitable conditions to promote cell reprogramming. In some embodiments, culturing the cell under suitable conditions to promote cell reprogramming comprises culturing the cell in a cell culture medium described herein.

In some aspects, provided herein are cells. In some embodiments, provided herein are bovine-induced pluripotent stem cells. In some embodiments, provided herein is a bovine-induced pluripotent stem cell produced by a method described herein.

Other aspects and embodiments of the disclosure will be apparent in light of the following detailed description and accompanying figures.

### BRIEF DESCRIPTION OF THE DRAWINGS

FIGS. 1A-IF show induction of biPSCs. FIG. 1A, left panel shows immunostaining of H3K9me3 expression in bMSCs infected with retroviral vector control or KDM4A. Bar=120 μm. FIG. 1A, right panel is a graph showing relative fluorescence intensity for H3K9me3 in bMSCs. Bar=mean±sd, n=3. Student's t-test was used for data analysis. FIG. 1B is a schematic showing an exemplary method of reprogramming of the bovine MSCs. FIG. 1C, upper panel are images showing development of iPSC colonies at day 7 and 17 of retroviral KdOSKMLN infection of bMSCs, bar=120 μm. FIG. 1C, lower panel are images showing picked biPSC colonies at P2 and P5, bar=250 μm. FIG. 1D are images showing expression of AP (upper panel) and SSEA4 (lower panel) in P2 biPSC colonies, bar=120 μm. FIG. 1E are images showing AP staining of biPSCs expanded in KT medium at P16. Left: Two AP-stained biPSC lines from one well of a 6-well plate. Right: AP-fluorescence from 2 biPSC lines under the microscope. Bar=120 μm. FIG. 1F shows results of qRT-PCR for transgene expression in four lines of biPSCs at passage 6 (P6), early passages (Lines 4-1 at P17, 4-6 at P17, 3-2 at P10), and later passages (Lines 4-1 at P43, 4-6 at P25, 3-2 at P29, O4 at P40). Transgene infected bMSCs at 48 h were used as the positive control. Bar=mean±sd, n=4. One way-ANOVA with Tukey's post hoc multiple comparison test was used for data analysis.

FIGS. 2A-2F show characterization of primed-like bovine iPSCs. FIG. 2A is an image showing flat colony morphology of 4-1 in KT medium. Bar=250 μm. FIG. 2B shows karyotype for O4 and 4-1 biPSC lines at passage 36 and 42, respectively. FIG. 2C shows qRT-PCR results for endogenous expression of pluripotent genes in four lines of biPSCs at early passages (4-1 at P17, 4-6 at P17, 3-2 at P10) and later passages (4-1 at P43, 4-6 at P25, 3-2 at P29, O4 at P40). Bar=mean±sd, n=4. One way-ANOVA with Tukey's post hoc multiple comparison test was used for data analysis. FIG. 2D shows representative immunostaining images of biPSCs in KT medium for OCT4, NANOG, SOX2, SSEA3, SSEA4, TRA-1-60. Bar=120 μm. FIG. 2E shows EBs formed from 4-1_KT, bar=625 μm. FIG. 2F shows immunostaining of differentiated cells for the three-germ layer markers (AFP for endoderm, SMA for mesoderm, and TUJ1 for ectoderm) after passaging of the EBs. bar=120 μm.

FIGS. 3A-3F show characterization of naïve-like bovine iPSCs. FIG. 3A shows flat, monolayered primed-like 4-1 biPSC colonies cultured in KT medium and passaged with collagenase (left), deteriorated colony morphology upon trypsinization (middle), and dome-shaped, naïve-like biPSCs cultured in TiF medium and passaged with trypsin (right), bar=250 μm. FIG. 3B is a graph showing cell proliferation difference between naïve and primed-like biPSCs. Mean±sd, n=3. Student's t-test was used for data comparison. FIG. 3C shows immunostaining of naïve-like 4-1 biPSCs in TiF medium with OCT4, NANOG, SOX2, SSEA3, SSEA4, TRA-1-60 antibodies. Bar=120 μm. FIG. 3D shows immunostaining of EBs derived from naïve-like biPSCs for three-germ layer markers (AFP for endoderm, SMA for mesoderm, and TUJ1 for ectoderm) after passaging of the EBs, bar=120 μm. FIG. 3E shows bisulfite sequencing results of bovine OCT4 distal enhancer (Left Panel) and proximal promoter (Right Panel) genomic regions. Open and closed circles represent unmethylated and methylated CpGs, respectively. The percentage of methylated CpG is shown at the bottom of each sample. FIG. 3F shows bisulfite sequencing results of bovine NANOG promoter genomic region. Open and closed circles represent unmethylated and methylated CpGs, respectively. The percentage of methylated CpG is shown at the bottom of each sample.

FIGS. 4A-4G show transcription analysis of biPSCs. FIG. 4A shows PCA analysis of RNA-seq data from bi PSCs in KT and TiF media, bESCs, and bovine embryos at the 16-cell and blastocyst stages. FIG. 4B shows a comparison of pluripotent gene expression between biPSCs, bESCs, bovine embryos, and bMSCs based on RNA-seq data. FIG. 4C shows Heatmap2 clustering analysis on 97 bovine pluripotent genes detected from RNA-seq data. FIG. 4D shows biological processes enriched in naïve-like biPSCs by GSEA analysis. FIG. 4E shows biological processes enriched in primed-like biPSCs by GSEA analysis. FIG. 4F is a graph showing significantly inhibited signaling pathways in naïve-like biPSCs (in TiF medium) compared with primed-like biPSCs (in KT medium). FIG. 4G shows Heatmap2 analysis on naïve- and primed-markers for biPSCs in TiF and KT media based on RNA-seq data.

FIGS. 5A-5D show mouse embryo chimerism analysis of biPSCs. FIG. 5A is an image showing DsRed-biPSCs incorporation of mouse blastocyst (right) at 24 h after embryo aggregation. Bar=60 μm. FIG. 5B shows DsRed-biPSCs contribution to the decidua of #4 (right) but not #1 (left) E8.5 mouse embryo. Bar=625 or 250 μm as indicated. FIG. 5C shows PCR analysis of DsRed-biPSC contribution in E8.5 mouse decidual tissue (upper panel) and embryo proper (EP, lower panel) using bovine-specific primers. FIG. 5D shows PCR analysis of DsRed-biPSC contribution on E8.5 embryos using pMXs-vector primers (upper panel). PCR using mouse-specific primers (lower panel) served as reference.

FIG. 6 shows blastocysts cloned from biPSCs. Blastocysts using bAF as nuclear donors (upper panel); GFP-Dox-biPSC as nuclear donors (middle panel), Dox was added into the embryo culture medium 6 days after activation of cloned embryos to induce the GFP expression; and DsRed-biPSC as nuclear donors (lower panel). Bar=100 μm.

FIGS. 7A-7C show generation of primary bovine MSCs. FIG. 7A shows pictures of primary human MSCs (hMSCs, from ATCC) and bovine MSCs (bMSCs) generated at passage 2 (p2). FIG. 7B shows qPCR comparison for marker gene expression in bMSCs (CD146, ITGA11) and bovine embryonic fibroblasts (bEFs) (CD10, CE26). Values are normalized with GAPDJ and relative to bEFs. FIG. 7C, left panel shows immunostaining of H3K9me2 expression in bMSCs infected with control vector or KDM4A. Bar=120 μm. FIG. 7C, right panel shows relative fluorescence intensity for H3K9me2 in BMSCs. Bar=mean+/−standard deviation, N=3. Students t-test was used for data analysis.

FIGS. 8A-8B show transcription analysis of biPSCs. FIG. 8A shows Heatmap 2 clustering analysis of 4,981 DEGs of different RNA-seq samples (FC>5, FDR<0.05). FIG. 8B shows additional significantly inhibited signaling pathways in naïve-like biPSCs (in TiF medium) compared with primed-like biPSCs (in KT medium).

FIG. 9 shows generation of fluorescent biPSCs. Left: bright field and DsRed fluorescence of biPSC colonies. Right: bright field and GFP fluorescence of biPSC colonies after Dox induction overnight. Bar=250 μm.

### DETAILED DESCRIPTION OF THE INVENTION

### 1. Definitions

To facilitate an understanding of the present technology, a number of terms and phrases are defined below. Additional definitions are set forth throughout the detailed description.

As used herein, the term “about,” when referring to a value or to an amount is meant to encompass variations of within ±20% from that value or amount. In some embodiments, “about” refers to ±20%, ±10%, ±5%, ±1%, ±0.5%, or ±0.1% from the specified amount, as such variations are appropriate.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “disruptor of telomeric silencing 1-like”, “Dot1-like”, “Dot1L”, “DOT1L”, or “histone methyltransferase DOT1L” are used interchangeably herein to refer to a methyltransferase found in humans as well as other eukaryotes. Dot1L is a member of the lysine methyltransferase family. Dot1L is not active on free histones and is the only methyltransferase that acts upon histone 3 (H3) lysine residue 79 (K79).

The term “WNT” or “Wnt” is used herein to describe any member of the Wnt family of proteins. The Wnt family of proteins are secreted, lipid-modified glycoproteins that regulate cell growth, function, and differentiation.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclature used in connection with, and techniques of, cell and tissue culture, biochemistry, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Compositions and Methods for Producing Bovine-Induced Pluripotent Stem Cells

Provided herein are bovine-induced pluripotent stem cells (biPSCs), along with compositions and methods for producing the same.

In some aspects, provided herein are compositions. In some embodiments, the compositions are cell culture media. In some embodiments, provided herein is a cell culture medium. In some embodiments, provided herein is a cell culture medium that may be used in methods for generating biPSCs.

In some embodiments, the cell culture medium comprises a WNT pathway inhibitor and a histone methyltransferase DOT1L inhibitor. Any suitable WNT pathway inhibitor and any suitable DOT1L inhibitor may be used. The terms “WNT pathway inhibitor”, “WNT signaling inhibitor”, and “WNT inhibitor” are used interchangeably herein to refer to an agent that inhibits one or more aspects of the WNT signaling pathway. In some embodiments, the WNT inhibitor impacts one or more components of the canonical Wnt/β-catenin signaling pathway. In some embodiments, the WNT inhibitor is a small molecule inhibitor. In some embodiments, the WNT inhibitor is selected from XAV-939, Wnt-C59, IWR1, ICG-001, IWP-2, LGK-974, CCT251545, FHF 535, IPW-4, JW 67, JW 74, KYA 1797K, NLS-StAx-H, TAK 715, WIC1, and derivatives thereof. In some embodiments, the WNT inhibitor is IWR1 or a derivative thereof. IWR1 is also referred to as IWR-1-endo, endo-IWR 1, or IRW-1 and has the structure:

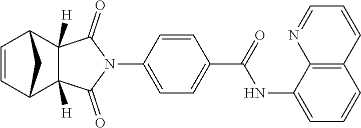 [SMILES: O=C(Nc1cccc2cccnc12)c1ccc(N2C(=O)[C@@H]3[C@H](C2=O)[C@@H]2C=C[C@H]3C2)cc1]

In some embodiments, the composition comprises more than one WNT inhibitor. For example, in some embodiments the composition comprises two, three, four, or more than four WNT inhibitors. In some embodiments, the composition comprises a combination of two or more WNT inhibitors selected from XAV-939, Wnt-C59, IWR1, ICG-001, IWP-2, LGK-974, CCT251545, FHF 535, IPW-4, JW 67, JW 74, KYA 1797K, NLS-StAx-H, TAK 715, WIC1, and derivatives thereof.

In some embodiments, the composition comprises the WNT inhibitor at a concentration of about 0.1 μM to about 25 μM. In some embodiments, the composition comprises the WNT inhibitor at a concentration of about 1 μM to about 20 μM. For example, in some embodiments, the composition comprises the WNT inhibitor at a concentration of about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM. In some embodiments, the composition comprises the WNT inhibitor at a concentration of about 0.1 μM to about 10 μM. In some embodiments, the composition comprises the WNT inhibitor at a concentration of about 0.5 to about 5 μM. In some embodiments, the composition comprises the WNT inhibitor at a concentration of about 2.5 μM. In some embodiments, the composition comprises the WNT inhibitor at a concentration of about 5 μM to about 15 μM. In some embodiments, the composition comprises the WNT inhibitor at a concentration of about 8 μM to about 12 μM. In some embodiments, the composition comprises the WNT inhibitor at a concentration of about 10 μM.

The term “DOT1L inhibitor” refers to an inhibitor of the histone methyltransferase DOT1L, which methylates H3K79 using S-adenosyl-L-methionine (SAM) as a co-factor. Any suitable DOT1L inhibitor may be used. In some embodiments, the DOT1L inhibitor is a SAM competitive inhibitor of DOT1L. In some embodiments, the DOT1L inhibitor is a small molecule inhibitor. In some embodiments, the DOT1L inhibitor is EPZ004777 (also referred to herein as “iDOT1L”) or a derivative thereof. EPZ004777 has the chemical name 7-[5-Deoxy-5-[[3-[[[[4-(1,1-dimethylethyl)phenyl]amino]carbonyl]amino]propyl](1-methylethyl)amino]-β-D-ribofuranosyl]-7H-pyrrolo[2,3-d]pyrimidin-4-amine Formic Acid Salt, and the structure:

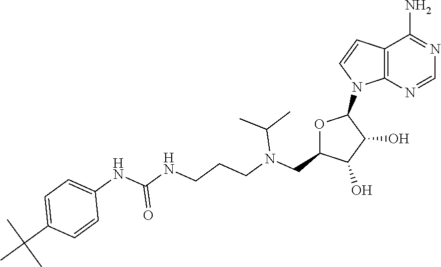 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

In some embodiments, the DOT1L inhibitor is EPZ-5676 or a derivative thereof. EPZ-5675 is also referred to as pinometostat, and has the chemical name (2R,3R,4S,5R)-2-(6-amino-9H-purin-9-yl)-5-((((1R,3S)-3-(2-(5-(tert-butyl)-1H-benzo[d]imidazol-2-yl)ethyl)cyclobutyl) (isopropyl)amino)methyl)tetrahydrofuran-3,4-diol and the structure:

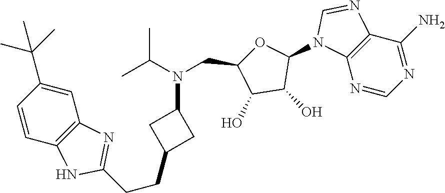

In some embodiments, the composition comprises more than one DOT1L inhibitor. For example, in some embodiments the composition comprises two DOT1L inhibitors. For example,

In some embodiments, the composition comprises the DOT1L inhibitor (e.g. EPZ004777, EPZ-5676) at a concentration of 0.5 μM to about 10 μM. in some embodiments, the composition comprises the DOT1L inhibitor at a concentration of 1 μM to about 5 μM. For example, in some embodiments the composition comprises the DOT1L inhibitor at a concentration of about 1 μM, about 2 μM, about 3 μM, about 4 μM, or about 5 μM. In some embodiments, the composition comprises the DOT1L inhibitor at a concentration of about 1 μM, about 1.5 μM, about 2 μM, about 2.5 μM, about 2.6 μM, about 2.7 μM, about 2.8 μM, about 2.9 μM, about 3 μM, about 3.1 μM, about 3.2 μM, about 3.3 μM, about 3.4 μM, about 3.5 μM, about 3.6 μM, about 3.7 μM, about 3.8 μM, about 3.9 μM, about 4 μM, about 5.5 μM, or about 5 μM. In some embodiments, the DOT1L inhibitor is EPZ004777.

In some embodiments, the cell culture medium further comprises human leukemia inhibitory factor (LIF). In some embodiments, the cell culture medium comprises about 100 U/mL to about 5000 U/mL human LIF. In some embodiments, the cell culture medium comprises about 500 U/mL to about 2000 U/mL human LIF. In some embodiments, the cell culture medium comprises about 100 U/ml, about 200 U/mL, about 300 U/ml, about 400 U/mL, about 500 U/ml, about 600 U/mL, about 700 U/ml, about 800 U/mL, about 900 U/ml, about 1000 U/mL, about 1100 U/ml, about 1200 U/mL, about 1300 U/ml, about 1400 U/mL, about 1500 U/ml, about 1600 U/mL, about 1700 U/ml, about 1800 U/mL, about 1900 U/ml, or about 2000 U/mL human LIF. In some embodiments, the cell culture medium comprises 1000 U/mL human LIF.

In some embodiments, the cell culture medium further comprises a suitable serum-free, commercially available stem cell culture medium, such as mTeSR™ or mTeSR™-plus. In some embodiments, the cell culture medium further comprises a synthetic serum-replacement medium, such as KSR medium.

In some embodiments, the cell culture medium comprising a WNT inhibitor and a DOT1L inhibitor is referred to herein as “KT medium”. In some embodiments, the KT medium comprises a stem cell culture medium (e.g. mTeSR, KSR medium, etc.), a DOTL inhibitor (e.g. EPZ004777, EPZ-5676), and a WNT inhibitor. Suitable WNT inhibitors are described above. In some embodiments, the KT medium comprises human LIF, such as at a concentration described above. In some embodiments, the WNT inhibitor is IWR1.

In some embodiments, the KT medium comprises about 0.5 μM to about 10 μM DOT1L inhibitor (e.g. EPZ004777, EPZ-5676) and about 1 μM to about 20 μM WNT inhibitor (e.g. IWR1). In some embodiments, the KT medium comprises 1 to about 5 μM DOT1L inhibitor (e.g. EPZ004777, EPZ-5676) and about 1 μM to about 20 μM WNT inhibitor (e.g. IWR1). In some embodiments, the KT medium comprises about 1 to about 5 μM DOT1L inhibitor (e.g. EPZ004777, EPZ-5676) and about 5 μM to about 15 μM WNT inhibitor (e.g. IWR1). In some embodiments, the KT medium comprises about 1 to about 5 μM DOT1L inhibitor (e.g. EPZ004777, EPZ-5676) and about 8 μM to about 12 μM WNT inhibitor (e.g. IWR1).

In some embodiments, the KT medium comprises about 1 to about 5 μM DOT1L inhibitor (e.g. about 1 μM, about 1.5 μM, about 2 μM, about 2.5 μM, about 3 μM, about 3.5 μM, about 4 μM, about 4.5 μM, or about 5 μM EPZ004777 or EPZ-5676) and about 5 μM to about 15 μM (e.g. about 5 μM, about 6 μM, about 7 μM, about 8 μM, about 9 μM, about 10 μM, about 11 μM, about 12 μM, about 13 μM, about 14 μM, about 15 μM) IWR1.

In some embodiments, the KT medium comprises about 1 μM to about 5 μM DOT1L inhibitor (e.g. EPZ004777, EPZ-5676) and about 8 μM to about 12 μM (e.g. about 8 μM, about 8.1 μM, about 8.2μ, about 8.3μ, about 8.4μ, about 8.5μ, about 8.6μ, about 8.7μ, about 8.8μ, about 8.9 μM, about 9.0 μM, about 9.1 μM, about 9.2 μM, about 9.3 μM, about 9.4 μM, about 9.5 μM, about 9.6 μM, about 9.7 μM, about 9.8 μM, about 9.9 μM, about 10.0 μM, about 10.1 μM, about 10.2 μM, about 10.3 μM, about 10.4 μM, about 10.5 μM, about 10.6 μM, about 10.7 μM, about 10.8 μM, about 10.9 μM, about 11.0 μM, about 11.1 μM, about 11.2 μM, about 11.3 μM, about 11.4 μM, about 11.5 μM, about 11.6 μM, about 11.7 μM, about 11.8 μM, about 11.9 μM, or about 12.0 μM) IWR1.

In some embodiments, the KT medium comprises about 3 to about 4 μM DOT1L inhibitor (e.g. about 3 μM, about 3.1 μM, about 3.2 μM, about 3.3 μM, about 3.4 μM, about 3.5 μM, about 3.6 μM, about 3.7 μM, about 3.8 μM, about 3.9 μM, about 4 μM EPZ004777 or EPZ-5676) and about 10 μM IWR1. In some embodiments, the KT medium comprises 3.3 μM EPZ004777 and about 10 μM IWR1.

In some embodiments, the DOT1L inhibitor is EPZ004777 and the WNT inhibitor is IWR1. In some embodiments, the KT medium comprises about 0.5 μM to about 10 μM EPZ004777 and about 1 μM to about 20 μM IWR1. In some embodiments, the KT medium comprises about 1 μM to about 5 μM (e.g. about 1 μM, about 1.5 μM, about 2 μM, about 2.5 μM, about 3 μM, about 3.5 μM, about 4 μM, about 4.5 μM, or about 5 μM) EPZ004777 and about 5 μM to about 15 μM (e.g. about 5 μM, about 6 μM, about 7 μM, about 8 μM, about 9 μM, about 10 μM, about 11 μM, about 12 μM, about 13 μM, about 14 μM, or about 15 μM) IWR1.

In some embodiments, the KT medium comprises about 1 μM to about 5 μM (e.g. about 1 μM, about 1.5 μM, about 2 μM, about 2.5 μM, about 3 μM, about 3.5 μM, about 4 μM, about 4.5 μM, or about 5 μM) EPZ004777 and about 8 μM to about 12 μM (e.g. about 8 μM, about 8.1 μM, about 8.2 μM, about 8.3μ, about 8.4μ, about 8.5μ, about 8.6μ, about 8.7μ, about 8.8μ, about 8.9μ M, about 9.0 μM, about 9.1 μM, about 9.2 μM, about 9.3 μM, about 9.4 μM, about 9.5 μM, about 9.6 μM, about 9.7 μM, about 9.8 μM, about 9.9 μM, about 10.0 μM, about 10.1 μM, about 10.2 μM, about 10.3μ, about 10.4μ, about 10.5μ, about 10.6μ, about 10.7μ, about 10.8μ, about 10.9μ M, about 11.0 μM, about 11.1 μM, about 11.2 μM, about 11.3 μM, about 11.4 μM, about 11.5 μM, about 11.6 μM, about 11.7 μM, about 11.8 μM, about 11.9 μM, or about 12.0 μM) IWR1.

In some embodiments, the KT medium comprises about 3 μM to about 4 μM (e.g. about 3 μM, about 3.1 μM, about 3.2 μM, about 3.3 μM, about 3.4 μM, about 3.5 μM, about 3.6 μM, about 3.7 μM, about 3.8 μM, about 3.9 μM, about 4 μM) EPZ004777 and about 8 μM to about 12 μM (e.g. about 8 μM, about 8.1 μM, about 8.2 μM, about 8.3 μM, about 8.4 μM, about 8.5 μM, about 8.6 μM, about 8.7 μM, about 8.8 μM, about 8.9 μM, about 9.0 μM, about 9.1 μM, about 9.2 μM, about 9.3 μM, about 9.4 μM, about 9.5 μM, about 9.6 μM, about 9.7 μM, about 9.8 μM, about 9.9 μM, about 10.0 μM, about 10.1 μM, about 10.2 μM, about 10.3 μM, about 10.4 μM, about 10.5 μM, about 10.6 μM, about 10.7 μM, about 10.8 μM, about 10.9 μM, about 11.0 μM, about 11.1 μM, about 11.2 μM, about 11.3 μM, about 11.4 μM, about 11.5 μM, about 11.6 μM, about 11.7 μM, about 11.8 μM, about 11.9 μM, or about 12.0 μM) IWR1. In some embodiments, the KT medium comprises about 3 μM to about 4 μM EPZ004777 and about 10 μM IWR1.

In some embodiments, the cell culture medium comprises a WNT inhibitor, a histone methyltransferase DOT1L inhibitor, human leukemia inhibitory factor (LIF), a glycogen synthase kinase 3 (GSK-3) inhibitor, a mitogen-activated protein kinase inhibitor, and an adenyl cyclase activator. Such a composition is referred to herein as “TiF” medium. In some embodiments, the TiF medium comprises the WNT inhibitor IWR1 and a DOT1L inhibitor selected from EPZ004777 and EPZ-5676.

In some embodiments, the cell culture medium (e.g. the TiF medium) comprises at least one glycogen synthase kinase 3 (GSK-3) inhibitor. In some embodiments, the GSK-3 inhibitor is a small molecule inhibitor. In some embodiments, the GSK-3 inhibitor is selected from CHIR-99021, CHIR-98014, LY2090314, BIO, TWS119, Tideglusib, SB216763, and derivatives thereof. In some embodiments, the GSK-3 inhibitor is CHIR-99021 or a derivative thereof. CHIR-99021 is also referred to as Laduviglusib, and has the structure:

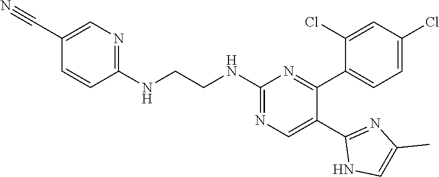 [SMILES: Cc1c[nH]c(-c2cnc(NCCNc3ccc(C#N)cn3)nc2-c2ccc(Cl)cc2Cl)n1]

In some embodiments, the cell culture medium comprises the GSK-3 inhibitor at a concentration of 0.5 μM to about 10 μM. In some embodiments, the composition comprises the GSK-3 inhibitor at a concentration of 1 μM to about 5 μM. For example, in some embodiments the composition comprises the GSK-3 inhibitor at a concentration of about 1 μM, about 2 μM, about 3 μM, about 4 μM, or about 5 μM. In some embodiments, the composition comprises the GSK-3 inhibitor at a concentration of about 1 μM, about 1.5 μM, about 2 μM, about 2.5 μM, about 2.6 μM, about 2.7 μM, about 2.8 μM, about 2.9 μM, about 3 μM, about 3.1 μM, about 3.2 μM, about 3.3 μM, about 3.4 μM, about 3.5 μM, about 3.6 μM, about 3.7 μM, about 3.8 μM, about 3.9 μM, about 4 μM, about 5.5 μM, or about 5 μM. In some embodiments, the GSK-3 inhibitor is CHIR-99021.

In some embodiments, the composition further comprises at least one mitogen-activated protein kinase inhibitor. In some embodiments, the at least one mitogen-activated protein kinase inhibitor inhibits MEK 1/2 (i.e. inhibits MEK1 and MEK2). In some embodiments, the MEK 1/2 inhibitor is selected from Trametinib, PD0325901, Selumetinib, UO126-EtOH, PD989059, and derivatives thereof. In some embodiments, the MEK1/2 inhibitor is PD0325901 or a derivative thereof PD0325901 is also referred to as Mirdametinib, and has the structure:

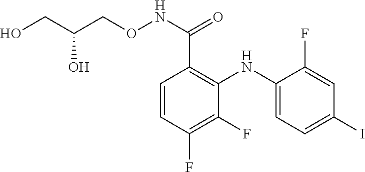 [SMILES: O=C(NOC[C@H](O)CO)c1ccc(F)c(F)c1Nc1ccc(I)cc1F]

In some embodiments, the composition comprises the MEK1/2 inhibitor at a concentration of about 0.1 μM to about 10 μM. In some embodiments, the composition comprises the MEK 1/2 inhibitor at a concentration of about 0.25 μM to about 7.5 μM. In some embodiments, the composition comprises the MEK 1/2 inhibitor at a concentration of about 0.5 μM to about 5 μM. In some embodiments, the composition comprises the MEK 1/2 inhibitor at a concentration of about 0.75 μM to about 2.5 μM. In some embodiments, the composition comprises the MEK 1/2 inhibitor at a concentration of about 1 μM. In some embodiments, the composition comprises the MEK1/2 inhibitor at a concentration of about 0.1 μM, about 0.2 μM, about 0.3 μM, about 0.4 μM, about 0.5 μM, about 0.6 μM, about 0.7 μM, about 0.8 μM, about 0.9 μM, about 1.0 μM, about 1.1 μM, about 1.2 μM, about 1.3 μM, about 1.4 μM, about 1.5 μM, about 1.6 μM, about 1.7 μM, about 1.8 μM, about 1.9 μM, about 2.0 μM, about 2.1 μM, about 2.2 μM, about 2.3 μM, about 2.4 μM, or about 2.5 μM. In some embodiments, the MEK1/2 inhibitor is PD0325901.

In some embodiments, the composition comprises at least one GSK-3 inhibitor and at least one MEK 1/2 inhibitor. In some embodiments, the composition comprises at least one GSK-3 inhibitor at a concentration described above and at least one MEK 1/2 inhibitor at a concentration described above.

In some embodiments, the cell culture medium further comprises a suitable serum-free, commercially available stem cell culture medium, such as mTeSR™ or mTeSR™-plus. In some embodiments, the composition comprises an adenyl cyclase activator. In some embodiments, the adenyl cyclase activator is Forskolin. In some embodiments, the composition comprises the adenyl cyclase activator (e.g. Forskolin) at a concentration of about 0.1 μM to about 20 μM. In some embodiments, the composition comprises the adenyl cyclase activator (e.g. Forskolin) at a concentration of about 1 μM to about 15 μM. In some embodiments, the composition comprises the adenyl cyclase activator (e.g. Forskolin) at a concentration of about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM. in some embodiments, the composition comprises about 10 μM Forskolin.

In some embodiments, the TiF medium comprises about the DOT1L inhibitor (e.g. EPZ004777, EPZ-5676) at a concentration of about 0.5 μM to about 10 μM. In some embodiments, the TiF medium comprises the DOT1L inhibitor (e.g. EPZ004777, EPZ-5676) at a concentration of 1 μM to about 5 μM. For example, in some embodiments the composition comprises the DOT1L inhibitor (e.g. EPZ004777, EPZ-5676) at a concentration of about 1 μM, about 2 μM, about 3 μM, about 4 μM, or about 5 μM. In some embodiments, the composition comprises the DOT1L inhibitor (e.g. EPZ004777, EPZ-5676) at a concentration of about 1 μM, about 1.5 μM, about 2 μM, about 2.5 μM, about 2.6 μM, about 2.7 μM, about 2.8 μM, about 2.9 μM, about 3 μM, about 3.1 μM, about 3.2 μM, about 3.3 μM, about 3.4 μM, about 3.5 μM, about 3.6 μM, about 3.7 μM, about 3.8 μM, about 3.9 μM, about 4 μM, about 5.5 μM, or about 5 μM. In some embodiments, the DOT1L inhibitor is EPZ004777.

In some embodiments, the TiF medium comprises the WNT inhibitor (e.g. IWR1) at a concentration of about 0.1 μM to about 10 μM. In some embodiments, the TiF medium comprises the WNT inhibitor (e.g. IWR1) at a concentration of about 0.5 μM to about 5 μM. In some embodiments, the composition comprises the WNT inhibitor (e.g. IWR1) at a concentration of about 2.5 μM. In some embodiments, the TiF medium comprises the WNT inhibitor (e.g. IWR1) at a concentration of about 0.1 μM, about 0.2 μM, about 0.3 μM, about 0.4 μM, about 0.5 μM, about 0.6 μM, about 0.7 μM, about 0.8 μM, about 0.9 μM, about 1.0 μM, about 1.1 μM, about 1.2 μM, about 1.3 μM, about 1.4 μM, about 1.5 μM, about 1.6 μM, about 1.7 μM, about 1.8 μM, about 1.9 μM, about 2.0 μM, about 2.1 μM, about 2.2μ, about 2.3μ, about 2.4μ, about 2.5μ, about 2.6μ, about 2.7μ, about 2.8μ, about 2.9 μM, about 3.0 μM, about 3.1 μM, about 3.2 μM, about 3.3 μM, about 3.4 μM, about 3.5 μM, about 3.6μ, about 3.7μ, about 3.8μ, about 3.9μ, about 4.0μ, about 4.1μ, about 4.2μ, about 4.3 μM, about 4.4 μM, about 4.5 μM, about 4.6 μM, about 4.7 μM, about 4.8 M, about 4.9 μM, or about 5.0 μM. In some embodiments, the WNT inhibitor is IWR1.

In some aspects, provided herein are methods for producing biPSCs. In some aspects, provided herein are biPSCs. In some embodiments, the biPSCs are produced by a method described herein.

In some embodiments, the method for producing biPSCs comprises reprogramming a bovine mesenchymal stem cell. In some embodiments, the method comprises providing a bovine mesenchymal stem cell, and inducing overexpression of a plurality of reprogramming factors in the bovine mesenchymal stem cell. In some embodiments, the plurality of reprogramming factors comprise lysine-specific demethylase 4A (KDM4A), OCT4, SOX2, KLF4, cMYC, LIN28, and NANOG. In some embodiments, overexpression of the plurality of reprogramming factors is achieved by transfecting the bovine mesenchymal stem cell. In some embodiments, the bovine mesenchymal stem cell is transfected with a suitable vector comprising sequences encoding the plurality of reprogramming factors. The vector can be any suitable vector, including viral vectors, plasmids, cosmids, phages, etc., In some embodiments, the bovine mesenchymal stem cell is transfected with a single vector loaded with each of the plurality of reprogramming factors. In some embodiments, the bovine mesenchymal stem cell is transfected with multiple vectors, each loaded with one or more of the desired reprogramming factors.

In some embodiments, the method further comprises culturing the mesenchymal stem cells under suitable conditions to promote cell reprogramming. For example, the cell may be cultured in a suitable medium to promote cell growth, survival, and reprogramming. In some embodiments, the cell is cultured in a first condition for a period of time or for a first number of passages, a second condition for a period of time or a second number of passages, a third condition for a period of time or a third number of passages, etc. In some embodiments, the cell is cultured in a first medium, which is switched to a second medium at a suitable point in time or after a suitable number of passages. In some embodiments, the total culture time is at least 12 days. For example, the total culture time may be at least 12 days, at least 13 days, at least 14 days, at least 15 days, at least 16 days, at least 17 days, or at least 18 days. In some embodiments, the total culture time is longer than 18 days. The medium may be switched at any suitable point in time in order to promote the desired cell reprogramming. In some embodiments, culturing the cell under suitable conditions to promote cell reprogramming comprises culturing the cell in a medium described herein, such as TiF medium and/or KT medium.

In some embodiments, culturing the cell under suitable conditions to promote cell reprogramming comprises culturing the cell in a medium comprising a WNT inhibitor and a DOT1L inhibitor for a first number of passages or a first duration of time, and culturing the cell in a medium comprising a WNT inhibitor, a histone methyltransferase DOT1L inhibitor, human leukemia inhibitory factor (LIF), a glycogen synthase kinase 3 (GSK-3) inhibitor, a mitogen-activated protein kinase inhibitor, and an adenyl cyclase activator for a second number of passages or a second duration of time. In some embodiments, culturing the cell under suitable conditions to promote cell reprogramming comprises culturing the cell in KT medium as described above for a first number of passages or a first duration of time, followed by culturing the cell in TiF medium as described for a second number of passages or a second duration of time. In some embodiments, prior to culturing the cell in TK medium the cell is cultured for a suitable duration of time in a growth medium. The growth medium can comprise any suitable growth medium used for promoting the growth and health stem cells, including MSC medium, KSR medium, DMEM/F12, and the like. In some embodiments, the growth medium comprises antibiotics, supplements, and/or cell apoptosis inhibitors. In some embodiments, the cells are cultured for 1-5 weeks following transfection (e.g. after inducing overexpression of the reprogramming factors) in the growth medium prior to initiating culture in the medium comprising the DOT1L inhibitor and the WNT inhibitor (e.g. the KT medium). In some embodiments, the cells are cultured for about 2-4 weeks in growth medium following inducing overexpression of the reprogramming factors and prior to culturing in the KT medium. In some embodiments, the cells are cultured for about 28 days prior to culturing in KT medium.

In some embodiments, the cells are cultured in KT medium for about 10-30 days (or a corresponding number of passages) prior to initiating culture in the TiF medium. The corresponding number of passages can be calculated based upon the doubling rate of the cells, as known in the art. Cells are typically passaged once confluency reaches roughly 80%. The cells may then be cultured in the TiF medium for a suitable number of days/passages. In some embodiments, the cells are cultured in KT Medium for about 10 days, about 12 days, about 14 days, about 16 days, about 18 days, about 20 days, about 21 days, about 22 days, about 23 days, about 24 days, about 25 days, about 26 days, about 27 days, about 28 days, about 29 days, or about 30 days (or a corresponding number of passages) in KT medium prior to initiating culture in the TiF medium. The cells may then be cultured in the TiF medium for a suitable duration (e.g. number of days or number of passages) until reprogramming has been achieved and bovine induced pluripotent stem cells are generated. Successful generation of bovine induced pluripotent stem cells (e.g. successful reprogramming) can be verified by measuring expression of genes/markers in the cells, including pluripotent markers such as OCT4, NANOG, LIN28A/B, SALL4, DNMT3A/3B.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

Overview: Pluripotent stem cells (PSCs) have been successfully developed in many species. However, the establishment of bovine-induced pluripotent stem cells (biPSCs) has been challenging. Described herein is the generation of biPSCs from bovine mesenchymal stem cells (bMSCs) by overexpression of lysine-specific demethylase 4A (KDM4A) and the other reprogramming factors OCT4, SOX2, KLF4, cMYC, LIN28, and NANOG (KdOSKMLN). These biPSCs exhibited silenced transgene expression at passage 10, and had prolonged self-renewal capacity for over 70 passages. The biPSCs have flat, primed-like PSC colony morphology in combined media of knockout serum replacement (KSR) and mTeSR, but switched to dome-shaped, naïve-like PSC colony morphology in mTeSR medium and 2i/LIF with single cell colonization capacity. These cells have comparable proliferation rate to the reported primed- or naïve-state human PSCs, with three-germ layer differentiation capacity and normal karyotype. Transcriptome analysis revealed a high similarity of biPSCs to reported bovine embryonic stem cells (ESCs) and embryos. The naïve-like biPSCs can be incorporated into mouse embryos, with the extended capacity of integration into extra-embryonic tissues. Finally, at least 24.5% cloning efficiency could be obtained in nuclear transfer (NT) experiment using late passage biPSCs as nuclear donors.

As the most common type of large, domesticated ungulates, bovine contributes to 45% of the global animal protein supply for human consumption [26]. The establishment of bona fide biPSCs will have huge impact on agricultural and biotechnological applications, to help establish a sustainable agriculture system to accommodate the need of an increasing global population. This technology is expected to produce abundant and renewable PSC resources in laboratory settings, to better understand embryogenesis in ruminants, to help generate genetically superior cattle with improved animal health, production and reproduction via genetic screening and manipulation [8,27], and also to promote the preclinical development of stem cell-based therapeutics using bovine disease models such as citrullinemia and leukocyte adhesion deficiency [24,28,29].

### Results

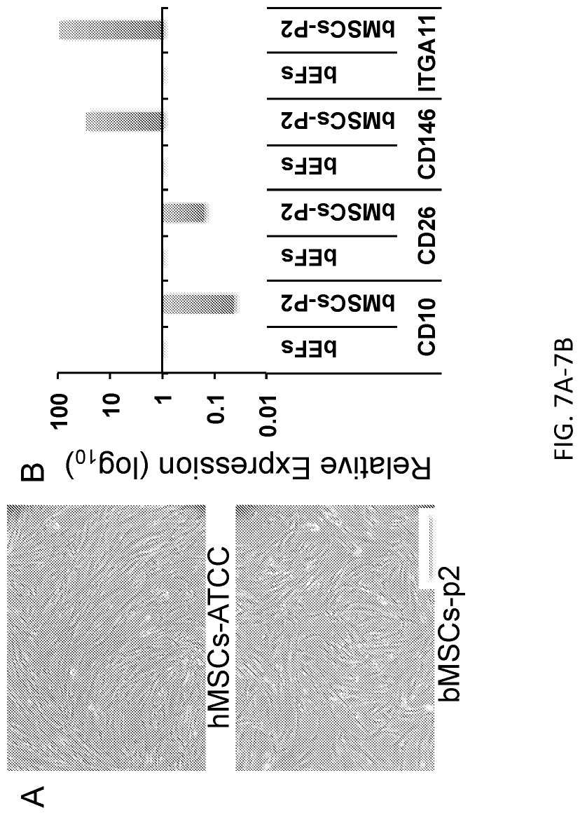

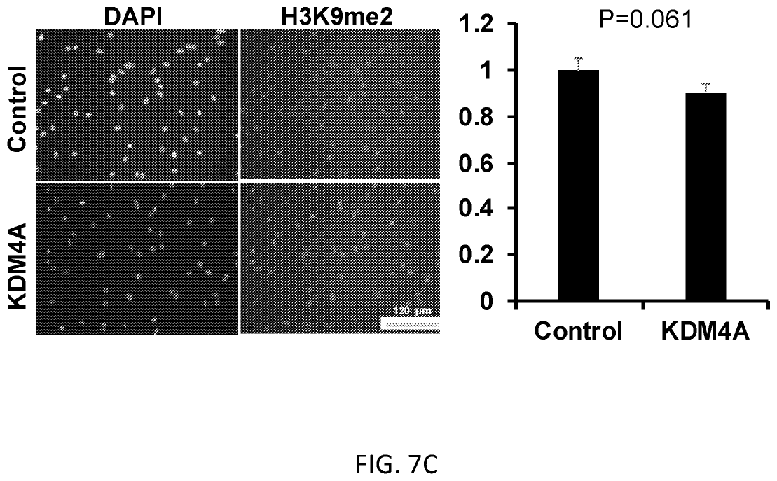

Establishment of bovine iPSCs: An iPSC induction scheme involving combined expression of human reprogramming factors OKMSLN (expressed in three different pMXs-retroviral vectors for O, KMS, and LN), together with the inhibitor of histone methyltransferase DOT1L (iDOT1L) and the WNT inhibitor (IWR1) was used to reprogram bovine primary mesenchymal stem cell-like cells (bMSCs) derived from bovine placenta (FIG. 7A,B). Although obvious cell aggregation was seen in the first two weeks of reprogramming, the development of any PSC-like colonies thereafter was not identified. Other attempts were made to reprogram bovine bMSCs in different PSC culture media, including the knockout serum replacement (KSR)-based ESC medium, mTeSR medium [31], and a modified epiblast stem cell (EpiSC) medium containing bFGF, Activin A, and WNT agonist CHIR-99021 (FAC) [32,33], but no PSC-like colonies were secured.

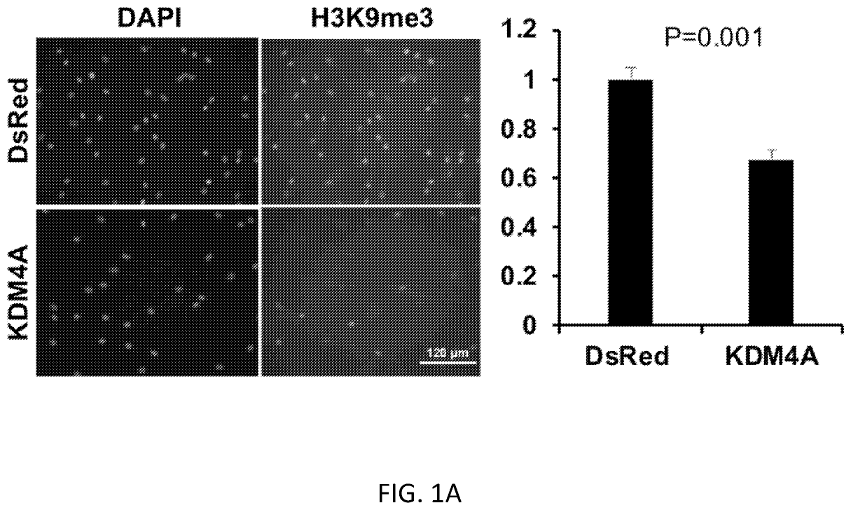

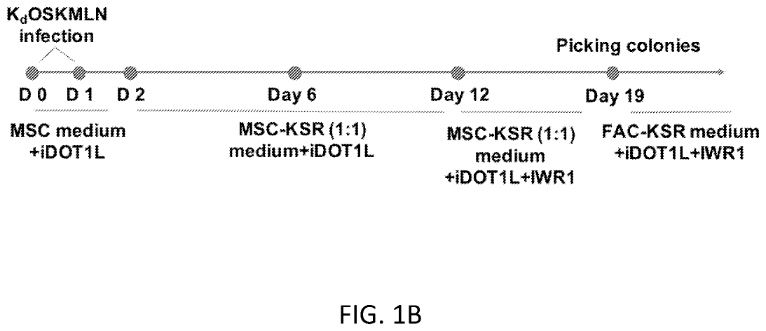

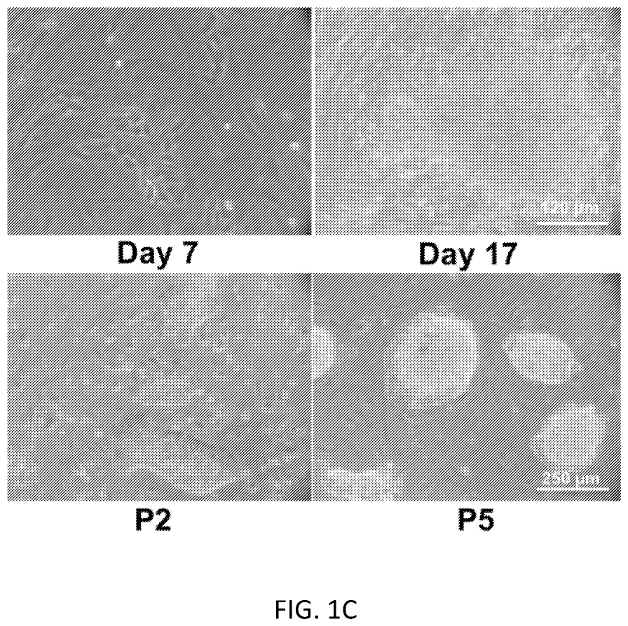

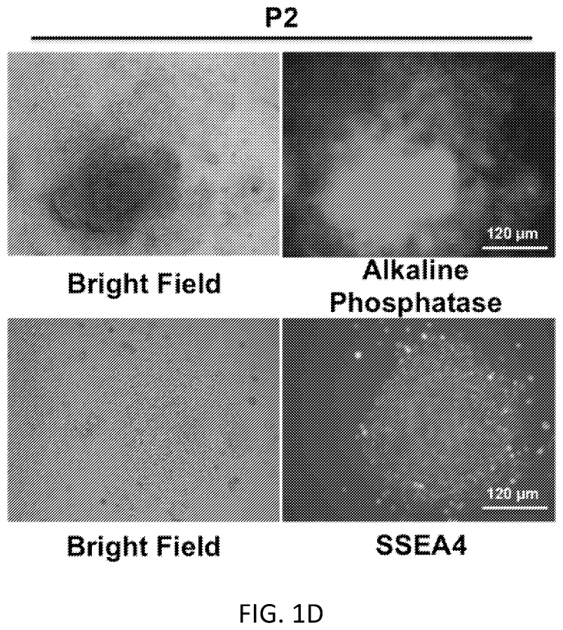

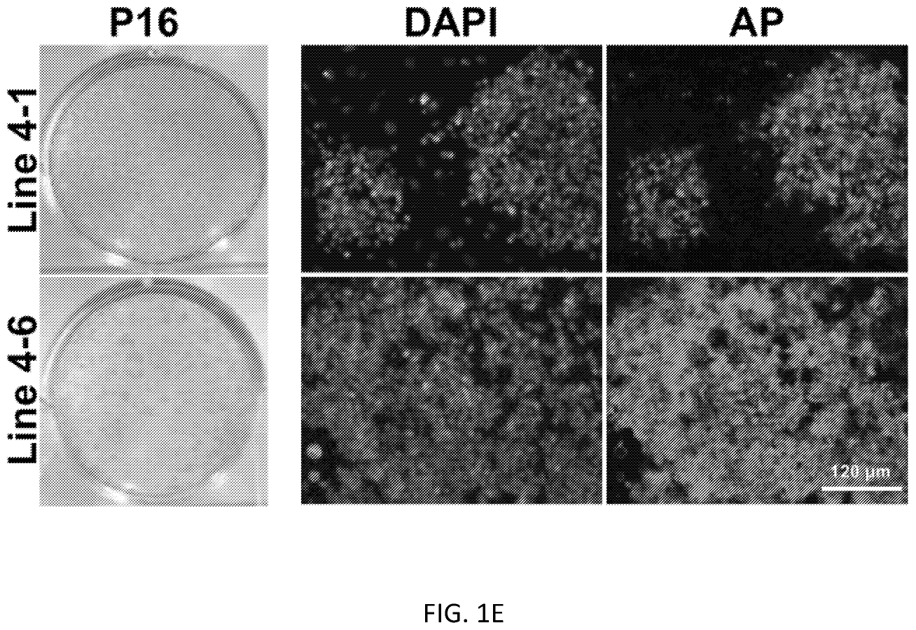

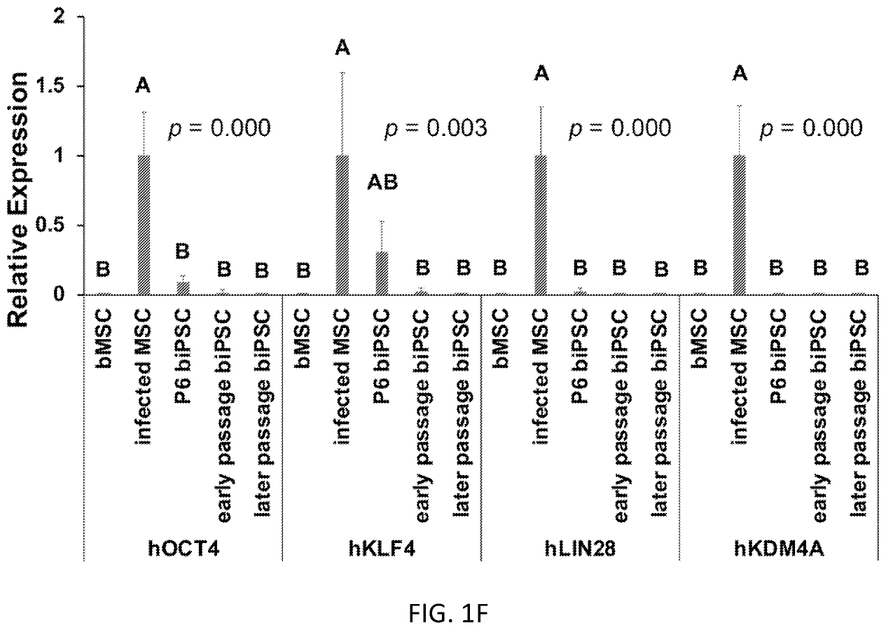

bMSCs overexpressing human KDM4A had reduced H3K9me3 but not H3K9me2 by immunostaining (FIGS. 1A and 7C). Based this evidence, pMXs-KDM4A was incorporated into the OSKMLN reprogramming (termed here KdOSKMLN) (FIG. 1B). Z-VAD-FMK, a pan caspase inhibitor, was also added to reduce the apoptosis of bMSC caused by retroviral infection (FIG. 1B). With the KdOSKMLN induction, PSC-like colonies were observed from the reprogrammed bMSCs on day 17 (FIG. 1C). The colonies were picked from day 19-30 and transferred to mitomycin C-treated mouse embryonic fibroblasts (MEF) feeders. They were cultured in a 1:1 combination of the FAC/KSR media (FIG. 1B,C) for the first three passages and thereafter in 1:1 combination of KSR/mTeSR media with the addition of iDOT1L and IWR1 (termed KT medium) (FIG. 1B). These bovine cells exhibited strong positive staining of the PSC surface markers alkaline-phosphatase (AP) and the stage-specific embryonic antigen 4 (SSEA4) at different passages (FIG. 1D,E).

The silence of exogenous transgenes is one of the key markers for successful reprogramming [1,38,39]. Four lines of the established biPSCs (Lines 4-1, 4-6, 3-2, and 04) were tested for the expression of viral transgenes. Since the KdOSKMLN transgenes were expressed either alone (O and Kd) or polycistronically (KMS and LN), specific PCR primers were designed to amplify DNA regions spanning the vector and the cloned human genes. qRT-PCR revealed that for all vectors, the expression of transgenes was inactivated in early passages (P10-P17) and remained silenced in later passages (P25-P43) (FIG. 1F).

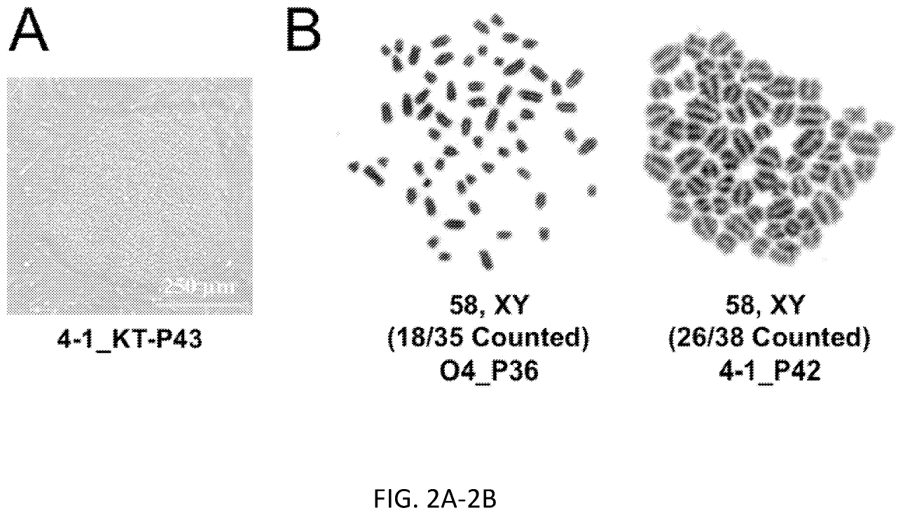

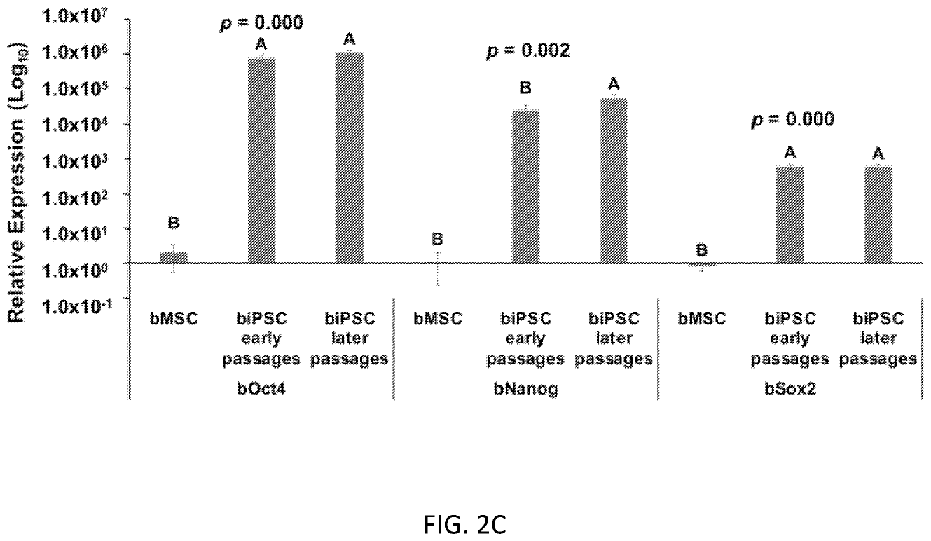

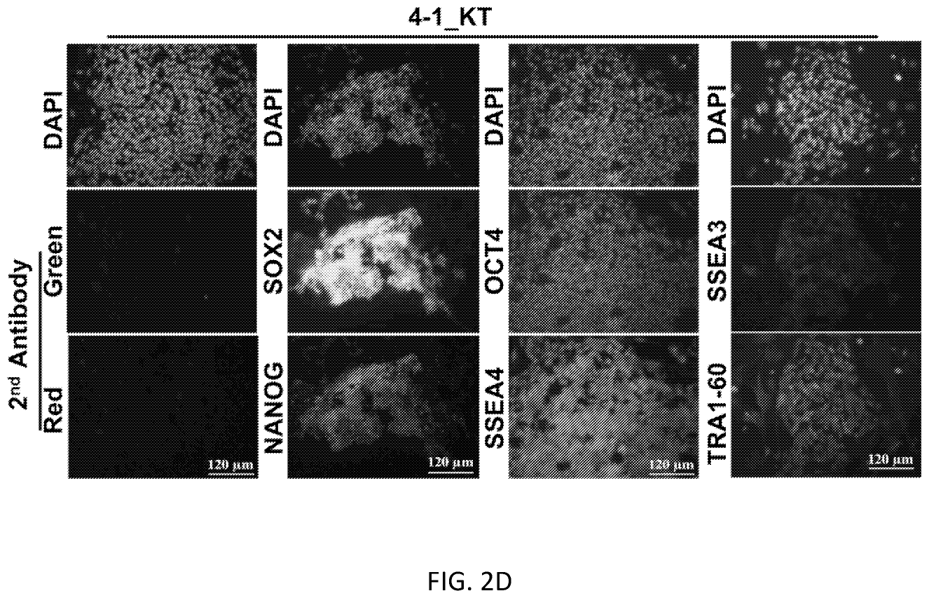

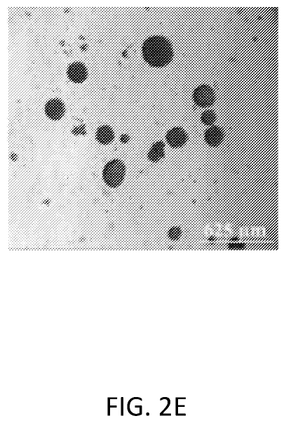

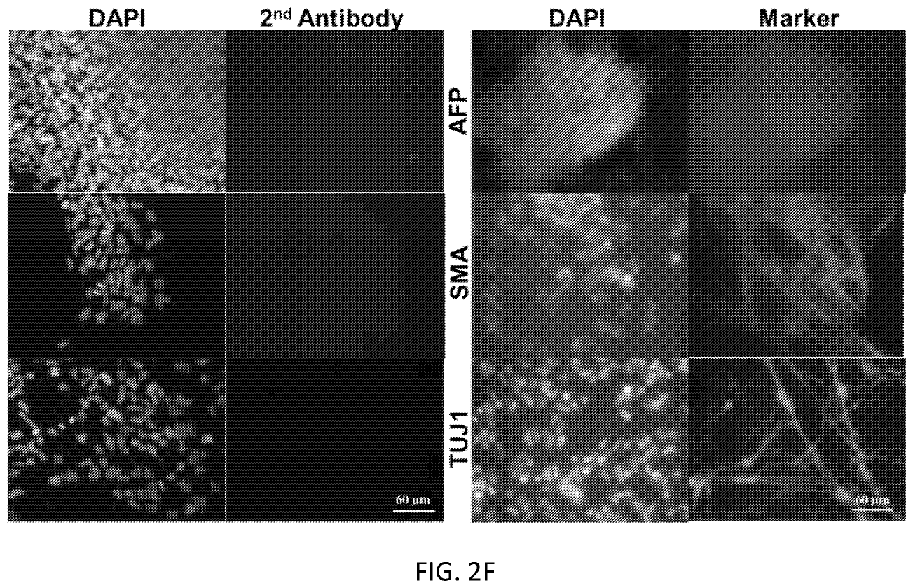

Characterization of Primed-Like Bovine iPSCs: The biPSCs cultured in KT medium could be passaged continuously with collagenase treatment, and displayed the monolayered, flat, and primed-PSC colony morphology (FIG. 2A). They exhibited normal karyotype (FIG. 2B). The activation of endogenous pluripotent genes was evaluated in these biPSCs. qRT-PCR of four biPSC lines using specific primers for endogenous key pluripotent genes revealed that bovine OCT4, NANOG, and SOX2 were highly activated in these biPSCs across different passages (FIG. 2C). Immunostaining using specific antibodies further confirmed the expression of these pluripotent proteins in biPSCs, together with the expression of additional pluripotent surface markers including SSEA4 and TRA-1-60, and a weak but distinguishable SSEA3 (FIG. 2D). The biPSCs also formed embryoid bodies (EBs) upon removal of the bFGF and culture in serum-containing medium (FIG. 2E), and differentiated into cell types expressing the three-germ layer specific markers (FIG. 2F).

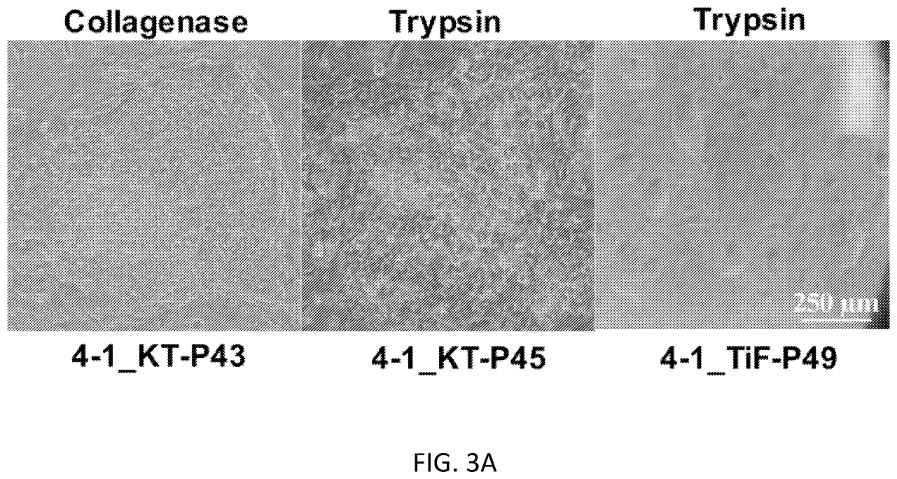

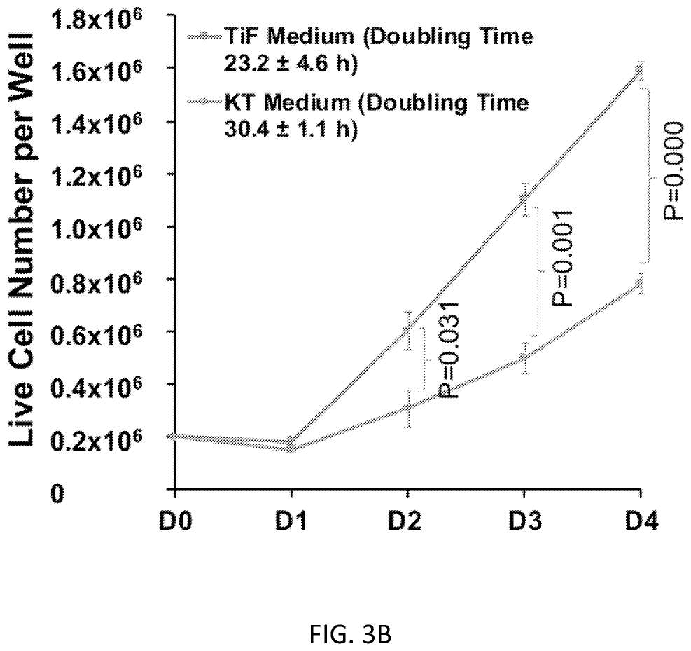

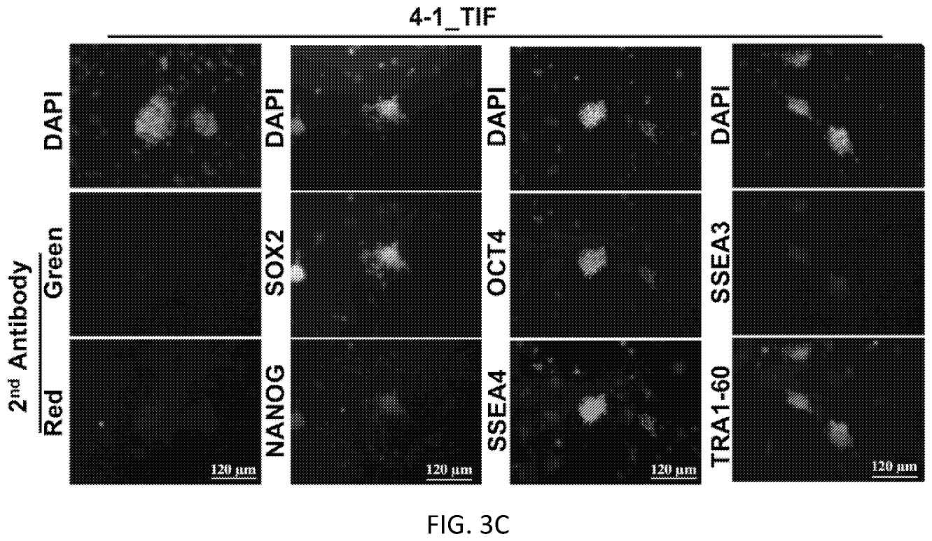

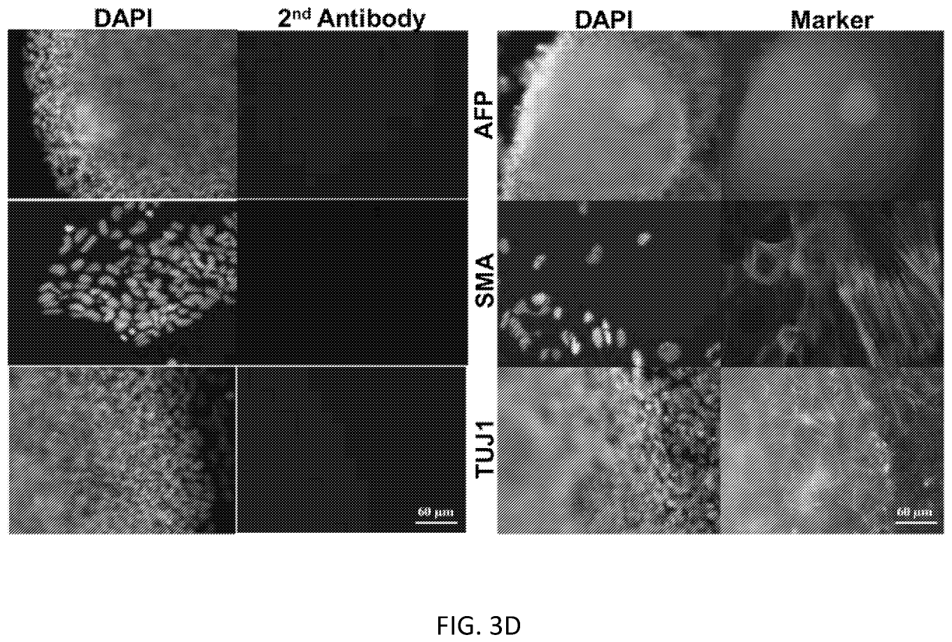

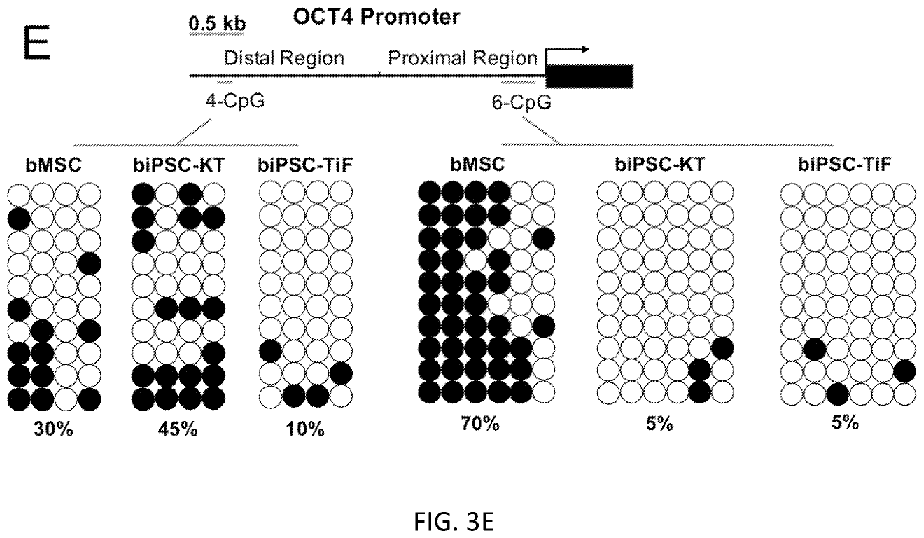

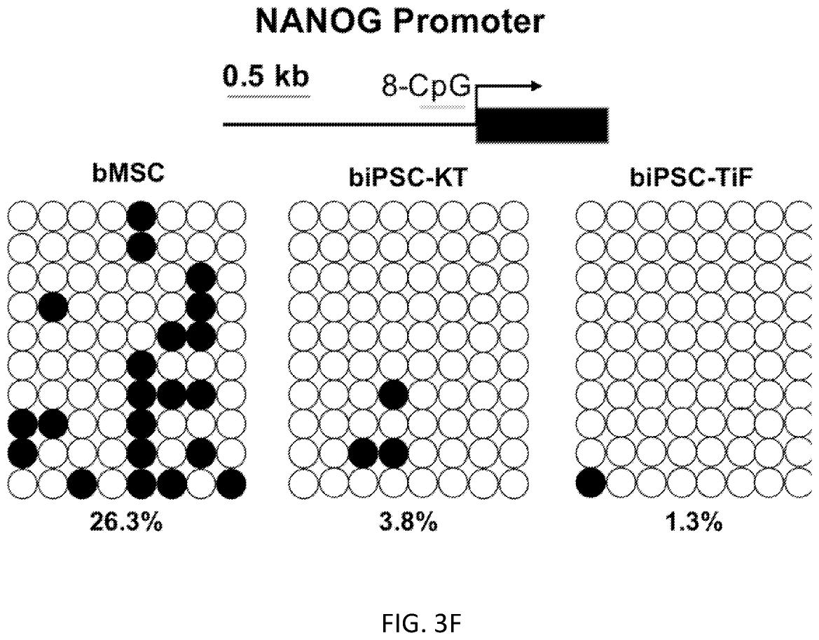

Development and Characterization of Naïve-like Bovine iPSCs: The primed-like biPSCs cultured in KT medium could not sustain single cell colonization and are refractory to trypsinization, which resulted in deterioration of colony morphology upon passaging (FIG. 3A). Accordingly, it was investigated whether these primed-like biPSCs could be converted to naïve-like PSCs in the medium containing extracellular signal-regulated kinases (ERK) 1/2 and glycogen synthase kinase (GSK) 3 inhibitors (PD0325901 and CHIR-99021) plus leukemia inhibitory factor (2i/LIF) [44,45]. After switching the KT medium into mTeSR medium supplemented with iDOT1L, IWR1, 2i/LIF, and the adenyl cyclase activator forskolin (turned here TiF medium), the round, dome-shaped naive-like PSC colonies appeared over the next couple of passages (FIG. 3A). These cells were capable of single cell colonization by trypsinization, a typical characteristic of naïve-state PSCs and valuable for genetic manipulation [46,47]. Cell proliferation assay revealed that the naïve-like biPSCs grew significantly faster than the primed-like cells, with a cell doubling time of 23.2 h compared to the 30.4 h for the primed-like biPSCs (FIG. 3B). These are similar to the previously reported doubling time of naïve (˜24 h) and primed (˜30 h) human PSCs, respectively [48,49]. The naïve-like biPSCs expressed pluripotent gene OCT4, SOX2, and NANOG, as well as pluripotent surface markers SSEA4 and TRA-1-60 similar to biPSCs cultured in KT medium (FIG. 3C). They formed EBs upon differentiation in serum-containing medium with cells expressing three-germ layer specific markers (FIG. 3D). Bisulfite sequencing to the bovine genomic DNA revealed highly demethylated bovine OCT4 (FIG. 3E, Right Panel) and NANOG (FIG. 3F) proximal promoter region in both naïve-like and primed-like biPSCs in contrast with the highly methylated bMSCs. Human and mouse naïve-state PSCs preferably activate the OCT4 distal enhancer than proximal prompter [50-55]. Notably, the distal enhancer region of OCT4 in naïve-like biPSCs is less methylated than bMSCs and primed-like biPSCs (FIG. 3E, Left Panel).

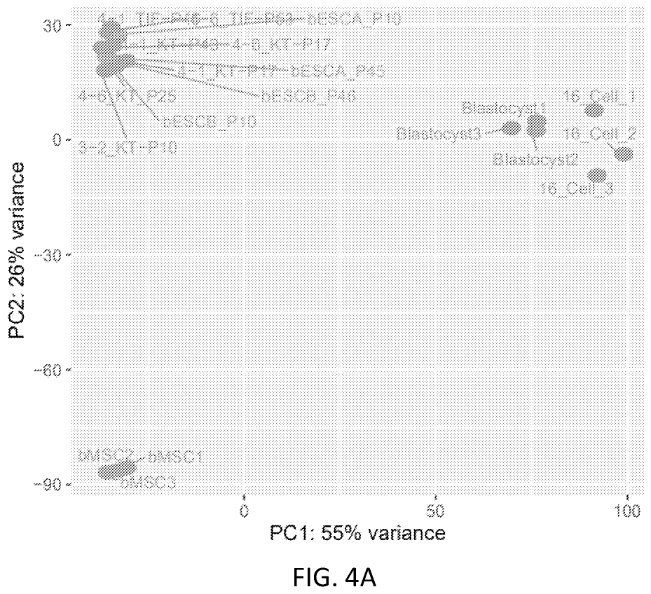

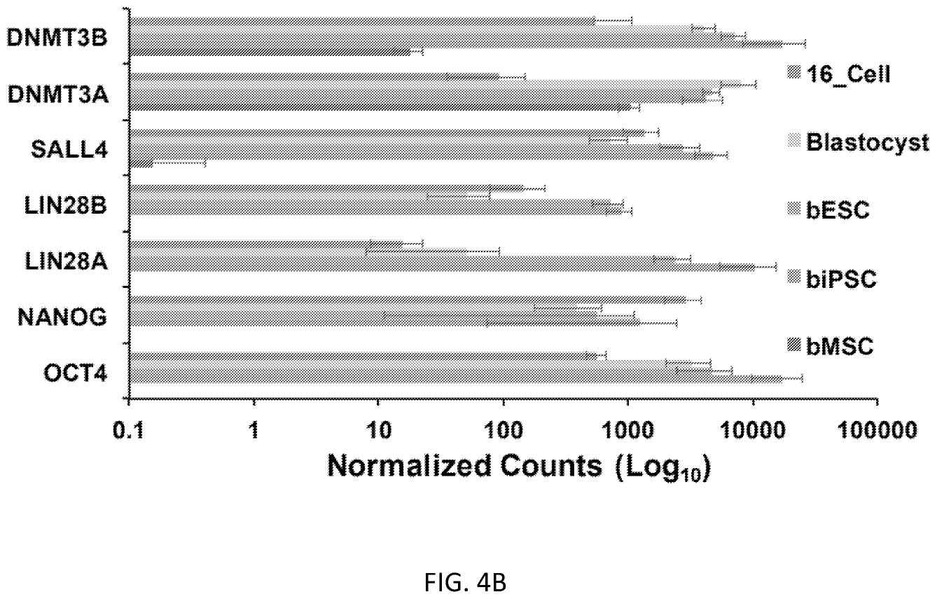

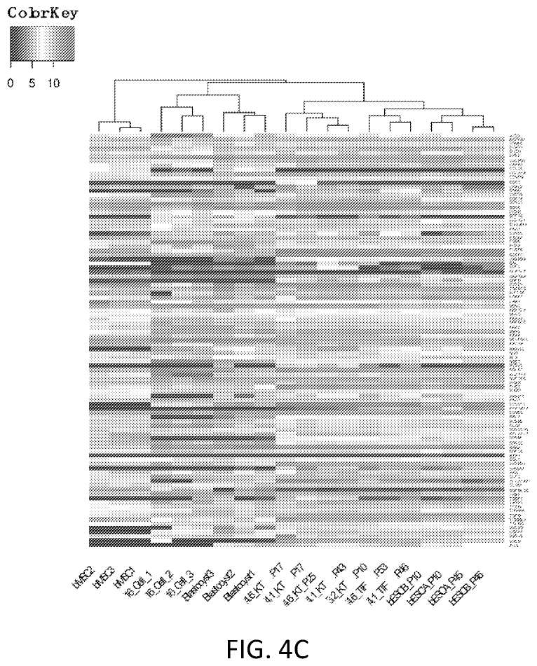

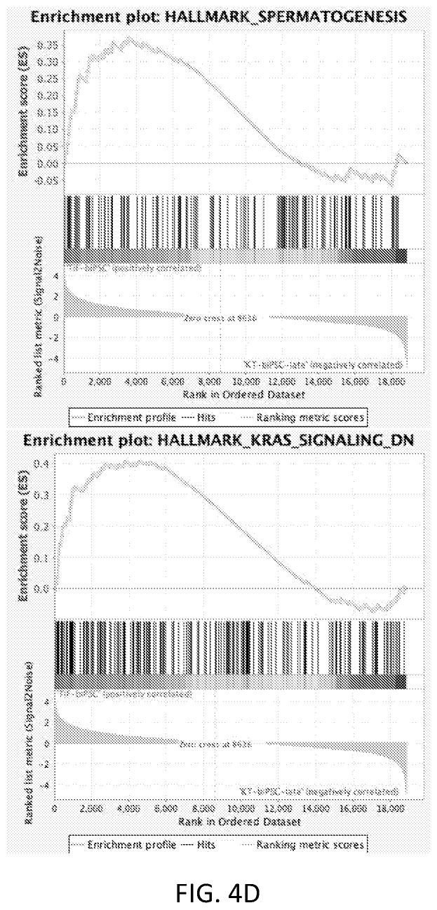

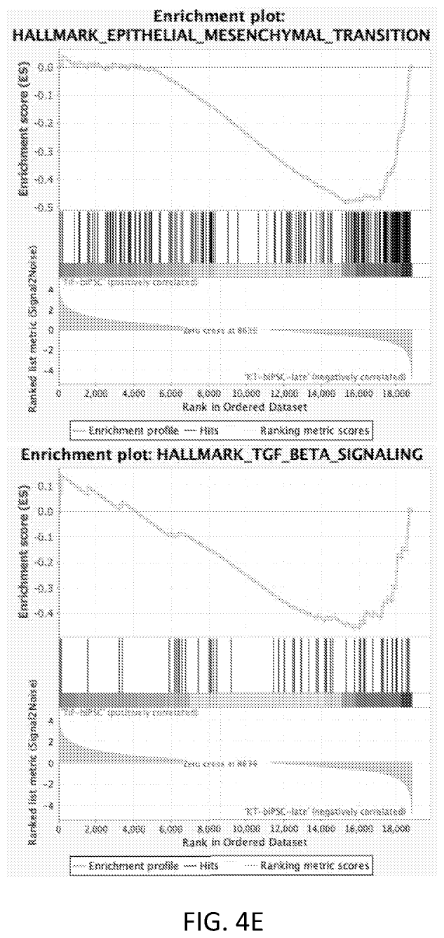

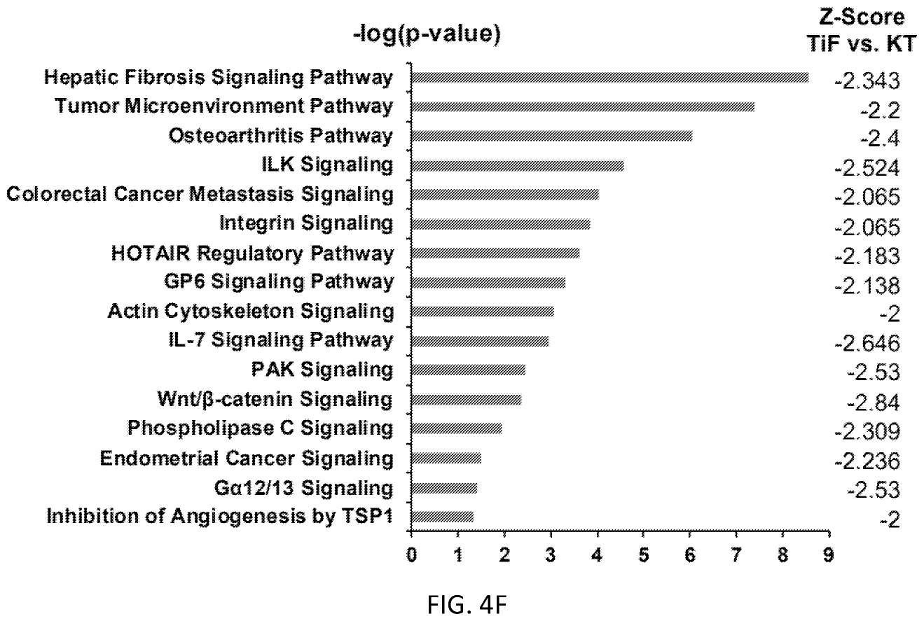

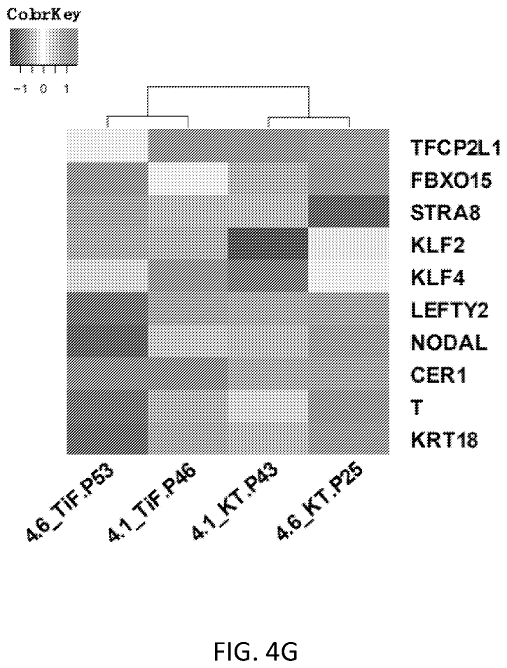

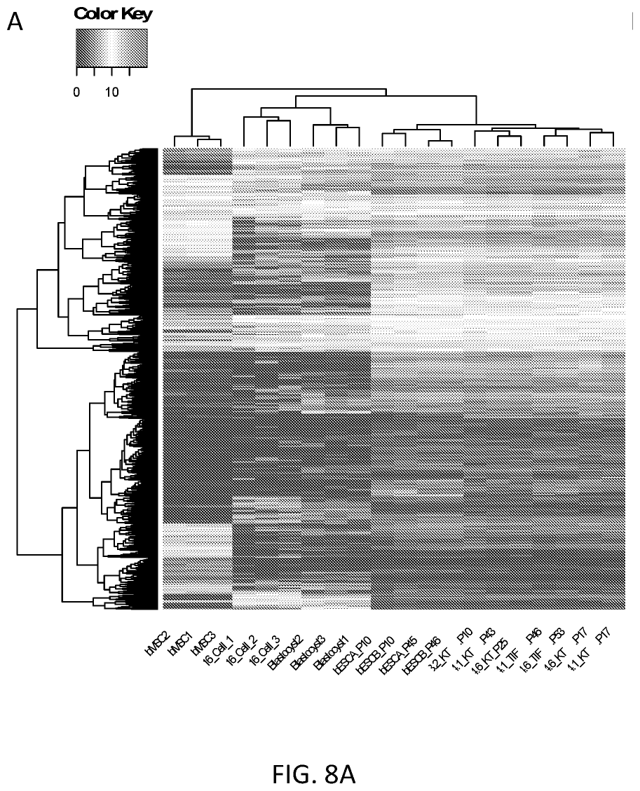

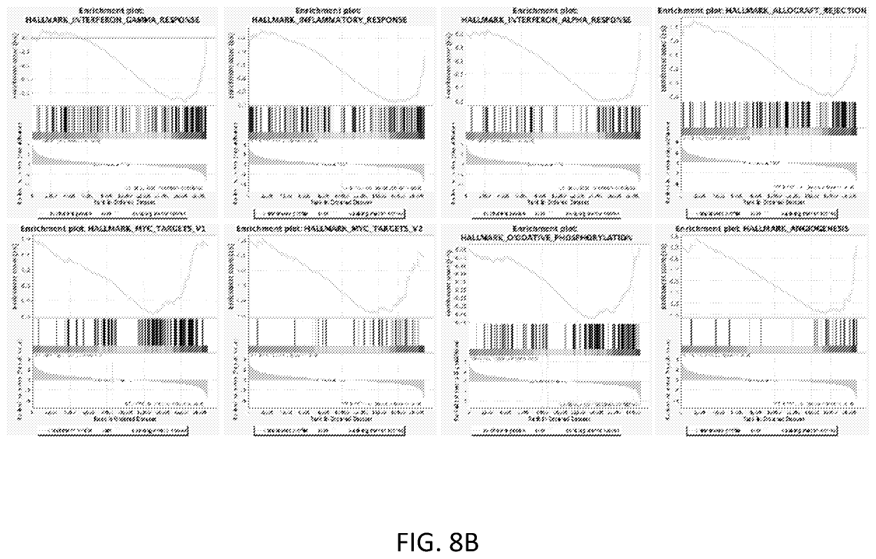

Global Transcriptome Analysis of bovine iPSCs: The global transcriptome profiles of the biPSC lines were analyzed and compared with the published data for the bovine embryos at the 16-cell and blastocyst stages [8,56], and bovine ESCs (bESCA/B from P10 to P46) [8] (GSE180931). Principle Component Analysis (PCA) revealed that the three lines of biPSCs in KT medium at different passages (4-1_KT-P17 and P43, 4-6_KT-P17 and P25, and 3-2_KT-P10) and two lines of biPSCs in TiF medium (4-1_TiF-P46, 4-6_TiF-P53) were clustered together with bovine ESCs (FIG. 4A). The biPSCs expressed pluripotent genes comparable to the bovine embryos and ESCs, including OCT4, NANOG, LIN28A/B, SALL4, DNMT3A/3B (FIG. 4B). Heatmap analysis for 4981 differentially expressed genes (DEGs) from bMSCs (absolute fold change (FC)>5, false discovery rate (FDR)<0.05) also revealed a high degree of similarity between the biPSCs and bovine embryos (FIG. 8A). Studies on human ESCs/iPSCs had identified a set of 169 pluripotent markers as a fingerprint for PSCs (StemCellDB) [57]. Data mining on our RNA-seq results identified 97 out of 100 annotated bovine genes orthologous to the StemCellDB fingerprint markers. Heatmap analysis on the expression of these 97 bovine genes again clustered all the biPSCs together with the bovine embryos and ESCs (FIG. 4C).

To gain a deeper understanding of cell signal changes between the two types of biPSCs, we performed gene set enrichment analysis (GSEA) to identify significantly enriched biological state/process gene-sets with the p-value<0.05 and FDR<0.25 [58-60]. The spermatogenesis and genes downregulated by KRAS signaling were found significantly enriched in naïve-like biPSCs (FIG. 4D), whereas 26 biological states/processes were found highly enriched in primed-like biPSCs, with the top ten of these including the epithelial-to-mesenchymal transition (EMT), MYC-targets, interferon and inflammatory responses, oxidative phosphorylation, angiogenesis, and TGF-β signaling FIG. 4E and FIG. 8B). We further used Ingenuity Pathway Analysis (IPA) to analyze the canonical signaling difference between naïve-like and primed-like biPSCs. The IPA regulation z-score algorithm was used to identify activated or inhibited biological functions (absolute z-score≥2). Sixteen pathways were found significantly inhibited in naïve-like biPSCs in TiF medium compared with primed-biPSCs in KT medium, including the hepatic fibrosis pathway, tumor microenvironment pathway, osteoarthritis pathway, etc. (FIG. 4F). Some of the signaling pathways correlated well with the biological processes identified in GSEA analysis, such as the colorectal cancer metastasis signaling vs. EMT, IL-7 signaling vs. interferon α/γ responses, osteoarthritis pathway vs. inflammatory response, etc. Therefore, inhibiting these pathways might be necessary to achieve naïve property from the primed-like biPSCs. Furthermore, comparing the expression of different pluripotentstage markers between the two types of biPSCs showed that the naïve-like biPSCs had increased expression of naïve-pluripotent markers including TFCP2L1, KLF2/4 [62], FBXO15, and STRA8 (54, 63), while the expression of primed-pluripotent markers including LEFTY2, NODAL, CER1, T [54, 63-65] and KRT18 were all downregulated (FIG. 4G).

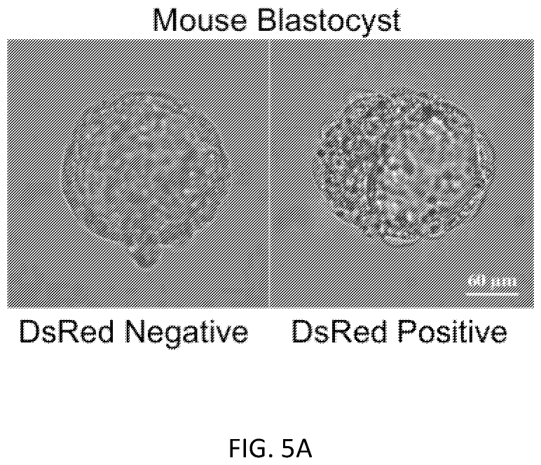

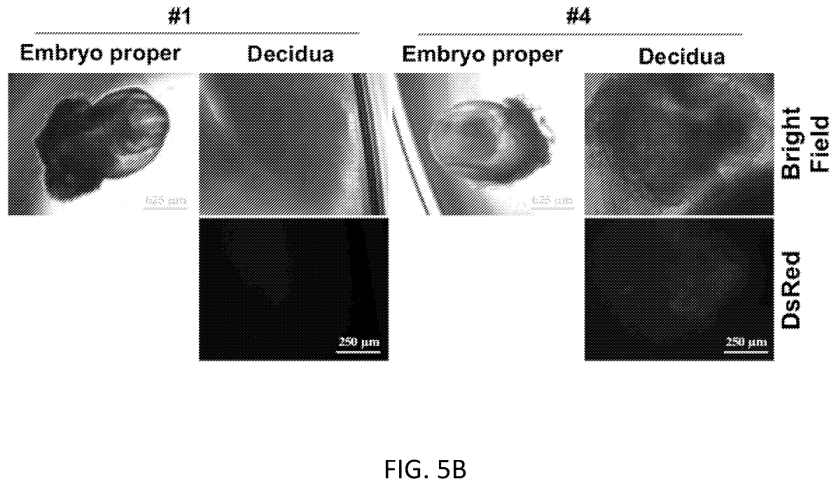

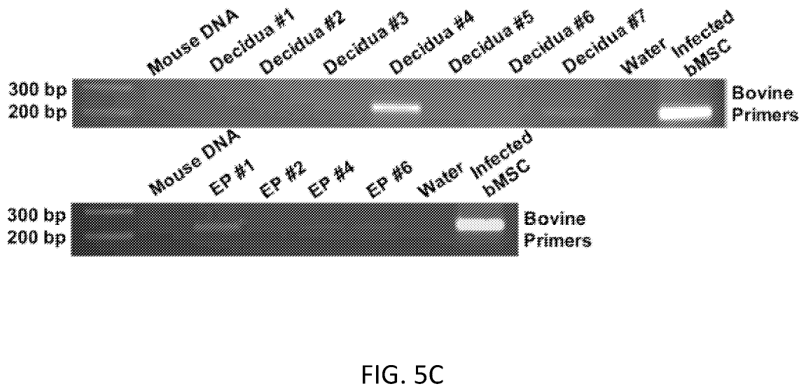

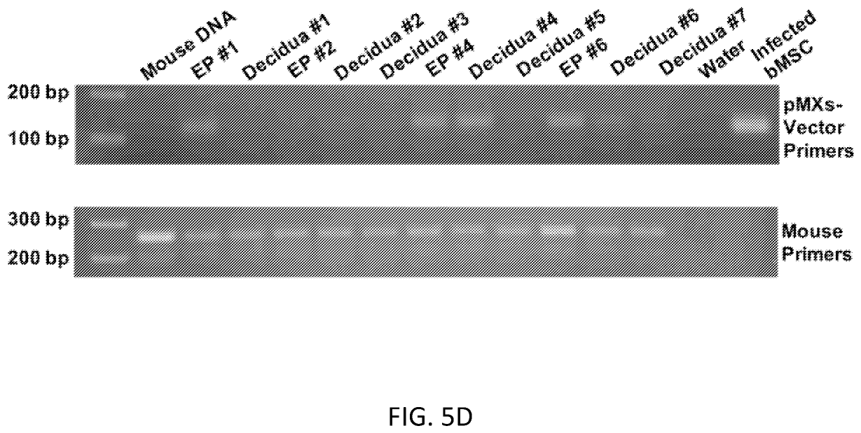

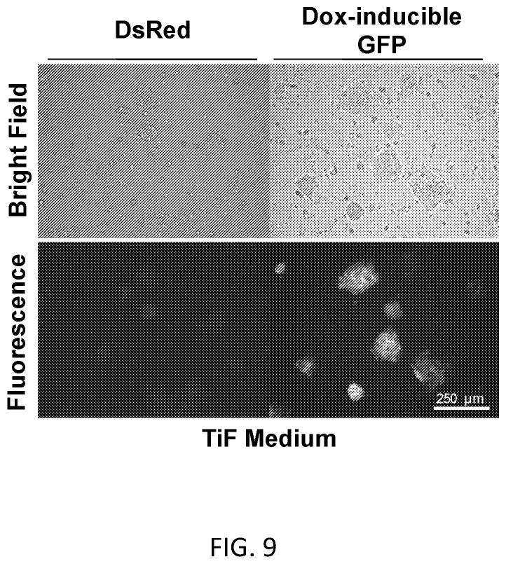

In Vivo Chimerism Capacity of Bovine iPSCs in Mouse Embryos: Using the naïve-like biPSCs, two biPSC lines with either constitutive expression of pMXs-DsRed or with Doxicyclin (Dox) inducible FUW-TetO-eGFP (DsRed or GFP-Dox-iPSCs) were derived. While the vast majority of the infected biPSC colonies rapidly silenced DsRed expression within two to three days, which was consistent with the observed transgene silencing property of these cells, several colonies were observed with faint but consistent DsRed expression. These colonies were expanded in TiF medium (FIG. 9). In order to evaluate the in vivo chimerism capacity of the biPSCs, an aggregation experiment was performed and early mouse morula (8-cell stage) were co-cultivated with the DsRed-biPSCs (P51). At 24 h after aggregation, six mouse embryos were fixed, which developed into early blastocysts, and the presence of red fluorescence in one blastocyst (FIG. 5A) was detected. The remaining blastocysts were then transferred into pseudopregnant female mice for further development. At E8.5, seven decidua were recovered with four containing well-developed embryos (#1, #2, #4, #6), whereas the other three were empty decidua likely due to embryo dysplasia. No red fluorescence was found in mouse embryo proper, but DsRed was observed in the decidual tissue harboring embryo #4 (FIG. 5B). This indicates that biPSCs were incorporated into trophoblasts or extraembryonic mesoderm leading to subsequent development into chorion tissue. It is possible that expression of DsRed in biPSCs might be too faint to detect, or subject to retroviral silencing after incorporation into ICM of early mouse embryos. To further verify biPSC chimerism, genomic DNA was extracted from mouse embryo proper and decidual tissue separately, and PCR was performed using bovine-specific primers to amplify bovine 1.715 satellite DNA. Genomic DNA isolated from mouse uterus and water were used as negative controls, and transgene infected bMSCs was used as positive control. Strong bovine-specific PCR product was detected in the decidual DNA of embryo #4, along with weak, but detectable PCR product in the decidua of embryos #6 and 7 (FIG. 5C, upper panel). Interestingly, PCR also detected positive band in the embryo propers #1, 4, and 6 (FIG. 5C, lower panel). The presence of biPSC DNA in the mouse embryo propers and decidual tissues was further confirmed by PCR using pMXs-vector specific prime pair (FIG. 5D). Therefore, it was concluded that the biPSCs are capable of contributing to mouse embryonic and extra-embryonic tissues.

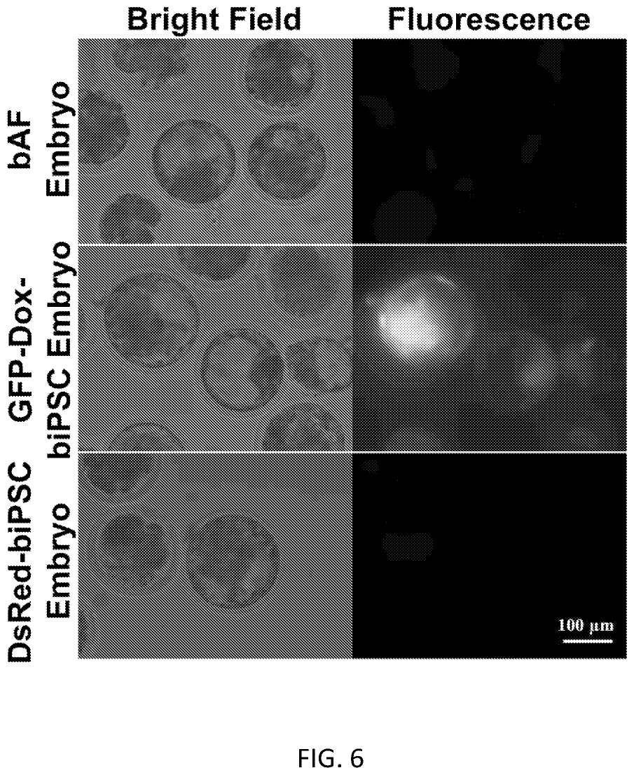

Efficiency of biPSCs as Donors for Somatic Cell Nuclear Transfer (SCNT): As the capacities of single cell colonization and infinite self-renewal make naïve-PSCs amenable for genetic modification and to serve as ideal nuclear donors for cloning, the efficiency of biPSCs was next evaluated in bovine NT experiments. Both late passage DsRed-biPSCs (P55) and GFP-Dox-biPSCs (P56) were used as nuclear donors to generate cloned embryos. No significant difference (p>0.05) either in fusion or cleavage rates was observed among two biPSC groups and a control group using bovine adult blastocysts (bAFs) as nuclear donors (Table 1). The two lines of biPSCs supported cloned blastocyst development at efficiencies of 24.7% (DsRed-biPSC) and 24.5% (GFP-Dox-biPSC), respectively (Table 1). There was no significant difference in blastocyst rates between the two biPSC lines and between biPSC and the control groups. When Dox was added on day six after embryo activation, clear GFP expression in the cloned GFP-Dox-biPSC blastocysts was observed (FIG. 6). However, no red fluorescence was detected in blastocysts derived from the DsRed-biPSCs, although expression of red fluorescence in biPSC colonies cultured prior to SCNT was observed. This is similar to what was observed in the mouse embryo aggregation experiment (FIG. 5B).

TABLE 1
Fusion Rates and In Vitro Developmental Capacities of Cloned
Bovine Embryos Generated Using biPSCs as Nuclear Donors.

| | | | No. of | No. of Cleaved | | No. of | |
|---|---|---|---|---|---|---|---|
| | No. of | No. of Fused | Reconstructed | Embryos | | Blastocysts | |
| Donor Cells | Oocytes | (mean ± sd %) | Embryos | (mean ± sd %) | | (mean ± sd %) * | |
| DsRed-biPSC | 194 | 170 (88.4 ± 5.4) ^{a} | 123 | 89 | (70.5 ± 17.0) ^{a} | 22 | (27.0 ± 9.0) ^{a} |
| GFP-Dox-biPSC | 206 | 183 (88.9 ± 1.9) ^{a} | 131 | 98 | (74.9 ± 10.8) ^{a} | 24 | (25.2 ± 15.2) ^{a} |
| bAF | 188 | 172 (91.5 ± 0.5) ^{a} | 125 | 111 | (88.6 ± 2.0) ^{a} | 45 | (39.3 ± 10.7) ^{a} |

* Blastocyst rates were calculated from the number of cleaved embryos.
^{a} No significant difference (p > 0.05) were observed among 3 groups in the same column. The data were analyzed by one-way ANOVA, n = 4.

### DISCUSSION

The generation of bovine iPSCs capable of long-term self-renewal and without transgene activation has been extremely challenging [24]. In this study, using a combination of seven factors (KdOSKMLN), and the reprogramming medium containing inhibitors to WNT (IWR1) and H3K79 methyltransferase Dot1L (iDot1L), the induction of primed-like biPSCs which do not rely on the continuous exogenous transgene activity for self-renewal was achieved. TiF medium was developed to convert the primed-like biPSCs to naïve-like biPSCs capable of single cell colonization. The primed and naïve-like biPSCs are both capable of propagation for at least 60 and 70 passages in the laboratory, respectively. These cells can differentiate into cells of the three-germ layers in vitro, and the naïve-biPSCs can incorporate into both mouse embryonic and extra-embryonic tissues in vivo. At high passage numbers (>P55), these biPSCs served as nuclear donor for NT experiment, and gave an average of 24.6% blastocyst development rate, which is comparable to the early-passage adult fibroblast donors. The blastocyst rate from high passage biPSCs here appears higher than the reported blastocyst rates using early passage bESCs as donors (10-21.2%) [8,25].

H3K9me3 modification in somatic cells represents an obstacle for iPSC generation. In bovine cells the KDM4A inhibits H3K9me3 but not H3K9me2 level. biPSCs were established herein using the six reprogramming factors plus KDM4A. The TiF medium described herein contains the MEK1 inhibitor PD0325901, which induce naïve-state PSCs by suppressing the activation of downstream ERK1/2 signaling [44,45].

The generation of completely reprogrammed biPSCs will provide invaluable PSC sources to facilitate both the basic research to understand cattle embryonic cell development and the applied studies to screen for genetic traits to improve reproduction, dairy/beef quality and productivity, and disease-resistance in cattle. Overall, described herein is a reprogramming system with KdOSKMLN factors that can be used to establish long-term passaged, transgene-silenced biPSCs.

### Materials and Methods

Chemicals, DNA Constructs, and Primary Bovine Cells: The DOT1L inhibitor EPZ004777 (iDOT1L) was purchased from AOBIOUS Inc. (Gloucester, MA, USA). WNT inhibitor IWR1, GSK-3 inhibitor CHIR-99021, MEK inhibitor PD0325901, ROCK inhibitor Y-27632 (ROCKi), and caspase inhibitor Z-VAD-FMK were purchased from Selleckchem (Houston, TX, USA). Forskolin was purchased from Fisher Scientific (Pittsburgh, PA, USA). Human reprogramming factors OCT4, SOX2, KLF4, MYC, NANOG, LIN28A, and KDM4A were used for bovine somatic cell reprogramming. The constructs pMXs-OCT4, FUW-TetO-eGFP, and FUW-m2rtTA were purchased from Addgene (Cambridge, MA, USA). Construction of the polycistronic vector pMXs-KLF4, MYC, and SOX2 (KMS) and pMXs-LIN28A and NANOG (LN) were described previously. Human KDM4A was cloned into pMXs-vector similarly to as previously described in [30]. Primary bovine umbilical cord-derived mesenchymal stem cells (bMSCs) were collected from Wharton's jelly part of the placenta of a newborn male Holstein calf, and were maintained with low serum MSC medium (ATCC, Manassas, VA, USA).

Reprogramming of bMSCs: For viral packaging, pMXs constructs were co-transfected into HEK293T cells with PUMVC and pCMV-VSV-G packaging plasmids, while FUW constructs were transfected with psPAX2 and pCMV-VSV-G plasmids (all from Addgene) using Fugene 6 (Promega, Madison, WI, USA) according to the protocol provided from the Addgene website. Virus-containing supernatant was harvested at 48 and 72 h post-transfection and filtered through 0.8 μm filters. Viral aliquots were stored at −70° C. until use. For reprogramming, on day minus one (−1), bMSCs were plated onto six-well tissue culture plates at a density of 5×105 cells/plate. On days 0 and 1, retrovirus carrying KdOSKMLN were added with 10 μg/mL polybrene (AmericanBIO, Natick, MA, USA). On day 2, the cells were maintained in a 1:1 mix of MSC medium and knockout serum replacement (KSR) medium, which contains 20% KSR in DMEM/F12, supplemented with 1×NEAA, 1× Glutamax, and 0.5× penicillin and streptomycin, 12 ng/mL human bFGF (all from Thermo Fisher Scientific, Waltham, MA, USA), and 1× β-mercaptoethanol (Merck Millipore, Billierica, MA, USA). The infected cells on day 5 were passaged onto mitomycin C treated MEF feeders in the presence of 10 μM ROCKi. On day 20, the medium was changed to a 1:1 mix of KSR medium and the FAC medium, which consists of a 1:1 mix of DMEM/F12 and neutral basal medium supplemented with 1× B27, 0.5× N2, 1×NEAA, 1× Glutamax, 0.5× penicillin and streptomycin, 1% KSR, and 0.05 μg/mL BSA. The 1:1 mix of KSR and FAC media is further supplemented with 12 ng/mL human bFGF (all from Thermo Fisher Scientific), 1× β-mercaptoethanol (Merck Millipore, Billierica, MA, USA), CHIR-99021 (3 μM), and Activin A (5 μg/mL). To inhibit the cell apoptosis during the viral infection and reprogramming, Z-VAD-FMK (20 μM) was supplemented in the medium during day 0 up to day 20. iDOT1L (3.3 μM) was applied in the media all the time, and IWR1 (10 μM) was added in reprogramming media since day 12. Starting from day 28, the biPSCs were maintained in a 1:1 mix of mTeSR-plus (STEMCELL Technologies, Cambridge, MA, USA) and KSR media supplemented with 1000 U/mL human LIF (Merck Millipore), 10 μM IWR1, 3.3 μM iDOT1L (KT medium) and passaged with collagenase. For some picked colonies, TiF medium was applied for reprogramming cells since passage 35, and passaged with trypsin or Tryple Express (Thermo Fisher Scientific). The TiF medium includes mTeSR plus medium, 1 μM PD0325901, 3 μM CHIR-99021, 1000 U/mL human LIF (Merck Millipore), 2.5 μM IWR1, 3.3 μM iDOT1L, and 10 μM Forskolin.

Quantitative Reverse Transcription-PCR (qRT-PCR) Analysis: Total RNAs were isolated from parental bMSCs, biPSCs, or differentiated cells with RNeasy mini kits (Qiagen, Hilden, Germany). Genomic DNAs were removed by DNase I (Qiagen) incubation. A total of 0.5 μg RNAs were then reverse transcribed into cDNA using iScript reverse transcription supermix (Bio-Rad Laboratories, Hercules, CA, USA). qRT-PCR reactions were performed with SYBR Green supermix (Bimake, Houston, TX, USA) using the ABI 7500 Fast platform (Thermo Fisher Scientific). GAPDH was used as the housekeeping gene for gene expression normalization. Data were processed with the software associated with ABI 7500.

Embryoid Body (EB) Differentiation: EB formation experiments were carried out with bovine iPSC lines. When growing to 70-80% confluency with mainly middle-size colonies, the cells were treated with freshly prepared 1 mg/mL collagenase for 30 min and removed from the plate by pipetting. After three washes with DMEM/F12, the cells were then plated onto low-adhesive petri dishes in EB formation medium on day 0 (KSR medium without bFGF). Half of the medium were changed to DMEM with 10% FBS every other day. EBs were treated by 0.05% Trypsin (Thermo Fisher Scientific) on day 5 and plated onto gelatin-coated plates. EBs at day 5 and day 14 were harvested for RNA isolation and gene expression analysis. The cells were subjected to immunofluorescence staining on days 12-14.

Immunostaining: For immunofluorescence, the cells were first fixed in 4% PFA for 15 min at room temperature. Following fixation, the cells were treated with 0.5% Triton X-100 in PBS for 15 min at room temperature for cell membrane permeabilization. After blocking with goat serum (Cell Signaling Technology, Danvers, MA, USA), the cells were incubated in goat serum containing primary antibodies for 2 h at 37° C., followed by secondary antibodies at room temperature for 1 h. Cells were counter-stained with DAPI and imaged under a Nikon fluorescence microscope. Primary antibodies including rabbit anti-OCT4 (Santa Cruz, CA, USA), rabbit anti-SOX2 (Merck Millipore), and mouse anti-NANOG (Merck Millipore) were used at 1:100 dilution while mouse anti-AFP (1:50; Cloud-clone Corp., TX, USA), rabbit anti-TUJ1 (1:500) and mouse anti-SMA (1:400) (Thermo Fisher Scientific) were diluted variously according to their manufacturer's instructions. Alexa Fluor 488 or 594 conjugated goat anti-rabbit or goat anti-mouse secondary antibody (Cell Signaling Technology) were used in 1:1000 dilution. For cell surface marker staining, the cells in different reprogramming conditions were stained with NL557-conjugated TRA-1-60 (1:100), SSEA-3 (1:50; R&D Systems, Minneapolis, MN, USA) and Alexa Fluor 594 conjugated SSEA-4 (1:100; BioLegend, San Diego, CA, USA) according to the manufacturer's protocol.

RNA-seq Data Analysis: Total RNA was isolated from reprogrammed cells with different treatments using RNeasy Mini kit (Qiagen). The quality of total RNA was examined by Nanodrop, Agarose Gel Electrophoresis, and the Agilent 2100 bioanalyzer. rRNA was then removed by using Ribo-Zero-rRNA Removal kit (Epicentre, Madison, WI, USA). First, the mRNA was fragmented randomly by adding fragmentation buffer, then the cDNA was synthesized by using mRNA template and random hexamers primer, after which a custom second-strand synthesis buffer (Illumina), dNTPs, RNase H, and DNA polymerase I were added to initiate the second-strand synthesis. Second, after a series of terminal repair, A ligation and sequencing adaptor ligation, the double-stranded cDNA library was completed through size selection and PCR enrichment. Finally, sequencing libraries were quantified by using Agilent 2100 bioanalyzer and then fed into Illumina sequencers. The RNA-seq data analysis was conducted at usegalaxy.org. Sequencing adapters and reads with low quality were trimmed using Cutadapt before mapping. The quality of reads after filtering was examined using fastQC. For mapping, bovine genomic sequence and RefSeq gene coordinate (ARS-UCD1.2/bosTau9) were downloaded from the UCSC genome browser. All filtered reads were aligned to bovine reference genome by RNA STAR (Galaxy Version 2.7.8a) with default parameters. The number of reads per gene was counted by feature Counts (Galaxy Version 2.0.1). Differentially expressed genes between different samples were identified using default parameters in DESeq2 (Galaxy Version 2.11.40.6+galaxy1), which generated a principal component analysis plot and the heatmap of the sample-to-sample distance matrix. The most differentially expressed genes (adjusted p-value<0.05) were extracted from DESeq2 results with an absolute fold change (FC)>5. The normalized counts for those differentially expressed genes and the Z-score of the counts were calculated on the galaxy platform and exhibited as heatmaps by Heatmap2 (Galaxy Version 3.0.1). The normalized counts for naïve- and primed-like biPSC samples were subjected to GSEA analysis (gsea-msigdb.org), with the log 2FC values used for IPA analysis (Qiagen).

Karyotyping: Karyotyping was carried out on biPSCs at different passages. biPSCs were first incubated with 10 μg/mL colcemid (Sigma-Aldrich, St. Louis, MO, USA) at 37° C. for 2 h, following which the cells were harvested by trypsinization. The cells were then incubated in hypotonic solution (0.56% KCl solution) for 15 min at 37° C. After three times washing in the fixative solution (methanol/glacial acetic acid 3:1), the cells were dropped onto wet and ice-cold glass slides. Giemsa (Sigma-Aldrich) at 1:20 dilution was applied onto the dried slides for staining. The nuclei were visualized with an Olympus microscope under a 100× oil objective lens.

Bisulfite Sequencing: For bisulfite sequencing, genomic DNAs were extracted and bisulfite converted using the EpiTeck Bilsulfite Kit (Qiagen). Bovine OCT4 and NANOG proximal promoter regions were amplified using PCR primers previously reported [10], and OCT4 distal primers were designed on MethPrimer [69]. The sequences of the primers are as follows. For OCT4 proximal promoter region primers, forward primer: 50-GTTTGGAGAGGGGTTTTGAAGAATGT GTAG-30 (SEQ ID NO: 1), and reverse primer: 50-ATCCCACCCACTAACCTTAACCTCTAAC-30 (SEQ ID NO: 2). For OCT4 distal enhancer region primers, forward primer: 50-TGTTTGGAGAATTTTATGGATAGAG-30 (SEQ ID NO: 3), and reverse primer: 50-CCAATTAAATCATCAAACCTAACTC-30 (SEQ ID NO: 4). For NANOG proximal promoter region primers, forward primer: 50-TAGGTGGTTATAGGAGATGTATTTTTG ATT-30 (SEQ ID NO: 5), and reverse primer: 50-TTATAAATAAAACTCAACCATACTTAACCC-30 (SEQ ID NO: 6). PCR were performed with Hot Start 2× Master Mix (Thermo Fisher Scientific) and cloned using the In-Fusion HD Cloning System into pIRES2-DsRed vector (Clontech) digested by BglII and EcoRI (New England Biolabs, Ipswich, MA, USA). Clones were picked, cultured in 6 mL LB medium with antibiotics overnight, and plasmid DNAs were extracted using a Qiaprep Mini Kit (Qiagen) and were subject to regular Sanger DNA sequencing. The results were then analyzed using the BiQ Analyzer [70].

Aggregation to Generate Chimeric Embryos: Chimeric embryos were generated by biPSC<−> mouse embryo aggregation as described previously [71]. Briefly, E2.5 morulae were isolated from CD-1 females (Charles River). Zona pellucidae were removed by brief exposure to acidic Tyrode's solution (Sigma T1788) followed by several washes in KSOM embryo medium (Millipore MR-101-D). Zona-free embryos were placed individually in micro-wells with KSOM embryo medium in an aggregation plate covered with light mineral oil (Fisher Cat #01211). Bovine iPSCs were fed with fresh culture media 2 h before aggregation. Cells were washed 2× with PBS, dislodged from the plates by brief exposure to 0.05% Trypsin (Sigma SM-2003-C) to obtain clumps of 8 to 12 cells. Two clumps of cells were then placed with a zona-free embryo in the micro-well and co-cultivated together in an 37° C. incubator with 6% CO2, 5% O2 and 89% N2. After an overnight incubation, biPSC<−> mouse embryo aggregates that did not develop into blastocyst were discarded and 20 to 25 blastocysts from each line were then transferred into pseudopregnant females for subsequent development. Embryos at various stages of gestation were harvested for analysis. For genotyping in mouse chimeras, genomic DNA was isolated from mouse embryos or extraembryonic tissue using DNeasy Blood & Tissue Kit (Qiagen). A total of 100 ng of genomic DNA was used for each PCR reaction with primers specific for mouse (forward primer: 50-TGTGGGCAAAGAGGCTTCAT-30 (SEQ ID NO: 7), reverse primer: 50-CAAAGCTGACTTAGCCTCAG-30 (SEQ ID NO: 8)), bovine (forward primer: 50-TGAGGCATGGAACTCCGCTT-30 (SEQ ID NO: 9), reverse primer: 50-GGTGGTTCCACATTCCGTAGGAC-30 (SEQ ID NO: 10)), and the pMXs-vector (forward primer: 50-CCGGTCGCTACCATTACCAG-30 (SEQ ID NO: 11), reverse primer: 50-CGGCCGCTCGAGTTTAAATA-30 (SEQ ID NO: 12)) sequences using Hot Start 2× Master Mix.

Nuclear Transfer Using biPSCs as Nuclear Donors: The biPSCs were cultured in a 6-well plate at 37° C., 5% CO2 in a humidified atmosphere. After culture for 4-5 days, the biPSCs forming typical compact colonies were used as nuclear donors for NT. Three hours before NT, biPSC culture medium was refreshed. Thirty minutes before NT, the biPSCs in one well of the 6-well plate were digested with 0.3 mL of Tryple Express (Cat. 12605-010, Gibco). The Tryple Express was neutralized by 5-fold dilution in the biPSC culture medium 8 min after digestion. The mixture was then centrifuged at 300×g for 5 min and the supernatant removed. The cell pellets were resuspended in 0.1 mL of the biPSC culture medium. The resuspended biPSCs were stored at room temperature (20-22° C.) before use. The bovine adult fibroblasts (bAFs) at passage 8-10 were grown to 80-90% confluence and used as nuclear donor cells in the control group after 24 h of serum starvation (0.5% FBS in DMEM). Bovine SCNT was performed as described by Fan et al. for goats [72], with modifications wherein an aspiration technique was used for oocyte recovery instead of a slicing technique and bovine oocyte maturation and culture media instead of caprine media. The bovine oocyte maturation medium consists of TCM-199 supplemented with 10% FBS, 5 μg/mL luteinizing hormone, 0.5 μg/mL follicle stimulating hormone, and 100 U/mL penicillin/streptomycin. The cloned embryos were in vitro cultured in bovine SOFaa medium with 5% FBS for 7 days after activation. On day 6, the cloned embryos derived from GFP-Dox-biPSCs were transferred into a prewarmed SOF-Dox drop, which consists of the SOF medium supplemented with 1 μg/mL of doxycycline, to induce the GFP expression. The in vitro development of cloned embryos was observed under a stereo microscope and GFP or DsRed expression detected by a fluorescent microscope (Observer Z1, Zeiss).

Statistical Analysis: One way-ANOVA with Tukey's multiple comparison post hoc test or Student's t-test was used for data analysis. The figures were presented as mean±standard deviation (sd). A p-value<0.05 (*) or 0.01 (**) was considered statistically significant. For NT experiment, the data from fusion and development of cloned embryos were analyzed using square arcsine transformation, followed by one-way analysis of variance (ANOVA). A p value of <0.05 for effects of factors was considered significant. A post hoc procedure with least significant difference (LSD) tests was used for multiple comparisons between groups. Means from differential staining were compared by one-way ANOVA (Jamovi). All the rest of the data were analyzed with SPSS platform.

Conclusions: Described herein is the successful generation of bovine iPSCs from somatic cells using the H3K9me3 demethylase KDM4A plus OSKMLN reprogramming factors. The established biPSCs exhibited silenced transgene expression with prolonged self-renewal capacity, high elevation of endogenous pluripotent factors, and displayed primed- or naïve-like pluripotent properties upon culturing in different medium conditions. The naïve-like biPSCs in high passage numbers contributed to both embryonic and extra-embryonic sections of mouse embryos, and could achieve a bovine blastocyst rate of at least 24.5% as nuclear donors in NT experiment. The established primed- and naïve-like biPSCs can serve as great resources for bovine embryology and biotechnology studies.

### REFERENCES

1. Okita, K.; Ichisaka, T.; Yamanaka, S. Generation of germline-competent induced pluripotent stem cells. Nat. Cell Biol. 2007, 448, 313-317.
2. Takahashi, K.; Yamanaka, S. Induction of Pluripotent Stem Cells from Mouse Embryonic and Adult Fibroblast Cultures by Defined Factors. Cell 2006, 126, 663-676.
3. Yu, J.; Vodyanik, M. A.; Smuga-Otto, K.; Antosiewicz-Bourget, J.; Frane, J. L.; Tian, S.; Nie, J.; Jonsdottir, G. A.; Ruotti, V.; Stewart, R.; et al. Induced pluripotent stem cell lines derived from human somatic cells. Science 2007, 318, 1917-1920.
4. Stice, S. L.; Strelchenko, N. S.; Keefer, C.; Matthews, L. Pluripotent bovine embryonic cell lines direct embryonic development following nuclear transfer. Biol. Reprod. 1996, 54, 100-110.
5. Gong, G.; Roach, M. L.; Jiang, L.; Yang, X.; Tian, X. C. Culture Conditions and Enzymatic Passaging of Bovine ESC-Like Cells. Cell. Reprogram. 2010, 12, 151-160.
6. Verma, V.; Huang, B.; Kallingappa, P. K.; Oback, B. Dual Kinase Inhibition Promotes Pluripotency in Finite Bovine Embryonic Cell Lines. Stem Cells Dev. 2013, 22, 1728-1742.
7. Maruotti, J.; Munoz, M.; Degrelle, S. A.; Gomez, E.; Louet, C.; Diez, C.; de Longchamp, P. H.; Brochard, V.; Hue, I.; Caamano, J. N.; et al. Efficient derivation of bovine embryonic stem cells needs more than active core pluripotency factors. Mol. Reprod. Dev. 2012, 79, 461-477.
8. Bogliotti, Y. S.; Wu, J.; Vilarino, M.; Okamura, D.; Soto, D. A.; Zhong, C.; Sakurai, M.; Sampaio, R.; Suzuki, K.; Belmonte, J. C. I.; et al. Efficient derivation of stable primed pluripotent embryonic stem cells from bovine blastocysts. Proc. Natl. Acad. Sci. USA 2018, 115, 2090-2095.
9. Deng, Y.; Liu, Q.; Luo, C.; Chen, S.; Li, X.; Wang, C.; Liu, Z.; Lei, X.; Zhang, H.; Sun, H.; et al. Generation of Induced Pluripotent Stem Cells from Buffalo (Bubalus bubalis) Fetal Fibroblasts with Buffalo Defined Factors. Stem Cells Dev. 2012, 21, 2485-2494.
10. Cao, H.; Yang, P.; Pu, Y.; Sun, X.; Yin, H.; Zhang, Y.; Zhang, Y.; Li, Y.; Liu, Y.; Fang, F.; et al. Characterization of Bovine Induced Pluripotent Stem Cells by Lentiviral Transduction of Reprogramming Factor Fusion Proteins. Int. J. Biol. Sci. 2012, 8, 498-511.
11. Sumer, H.; Liu, J.; Malaver-Ortega, L. F.; Lim, M. L.; Khodadadi, K.; Verma, P. J. NANOG is a key factor for induction of pluripotency in bovine adult fibroblasts1. J. Anim. Sci. 2011, 89, 2708-2716.
12. Han, X.; Han, J.; Ding, F.; Cao, S.; Lim, S. S.; Dai, Y.; Zhang, R.; Zhang, Y.; Lim, B.; Li, N. Generation of induced pluripotent stem cells from bovine embryonic fibroblast cells. Cell Res. 2011, 21, 1509-1512.
13. Talluri, T. R.; Kumar, D.; Glage, S.; Garrels, W.; Ivics, Z.; Debowski, K.; Behr, R.; Niemann, H.; Kues, W. A. Derivation and Characterization of Bovine Induced Pluripotent Stem Cells by Transposon-Mediated Reprogramming. Cell. Reprogram. 2015, 17, 131-140.
14. Malaver-Ortega, L. F.; Sumer, H.; Liu, J.; Verma, P. J. Inhibition of JAK-STAT ERK/MAPK and Glycogen Synthase Kinase-3 Induces a Change in Gene Expression Profile of Bovine Induced Pluripotent Stem Cells. Stem Cells Int. 2016, 2016, 5127984.
15. Kawaguchi, T.; Tsukiyama, T.; Kimura, K.; Matsuyama, S.; Minami, N.; Yamada, M.; Imai, H. Generation of Naïve Bovine Induced Pluripotent Stem Cells Using PiggyBac Transposition of Doxycycline-Inducible Transcription Factors. PLOS ONE 2015, 10, e0135403.
16. Heo, Y. T.; Quan, X.; Xu, Y. N.; Baek, S.; Choi, H.; Kim, N.-H.; Kim, J. CRISPR/Cas9 Nuclease-Mediated Gene Knock-In in Bovine-Induced Pluripotent Cells. Stem Cells Dev. 2015, 24, 393-402.
17. Lin, Y.-C.; Kuo, K.-K.; Wuputra, K.; Lin, S.-H.; Ku, C.-C.; Yang, Y.-H.; Wang, S.-W.; Wang, S.-W.; Wu, D.-C.; Wu, C.-C.; et al. Bovine Induced Pluripotent Stem Cells Are More Resistant to Apoptosis than Testicular Cells in Response to Mono-(2-ethylhexyl) Phthalate. Int. J. Mol. Sci. 2014, 15, 5011-5031.
18. Wang, S.-W.; Wang, S. S.-W.; Wu, D.-C.; Lin, Y.-C.; Ku, C.-C.; Wu, C.-C.; Chai, C.-Y.; Lee, J.-N.; Tsai, E.-M.; Lin, C.-L.; et al. Androgen receptor-mediated apoptosis in bovine testicular induced pluripotent stem cells in response to phthalate esters. Cell Death Dis. 2013, 4, e907.
19. Bressan, F. F.; Bassanezze, V.; Pessoa, L. V. D. F.; Sacramento, C. B.; Malta, T. M.; Kashima, S.; Neto, P. F.; Strefezzi, R. D. F.; Pieri, N. C. G.; Krieger, J. E.; et al. Generation of induced pluripotent stem cells from large domestic animals. Stem Cell Res. Ther. 2020, 11, 247.
20. Pillai, V. V.; Kei, T. G.; Reddy, S. E.; Das, M.; Abratte, C.; Cheong, S. H.; Selvaraj, V. Induced pluripotent stem cell generation from bovine somatic cells indicates unmet needs for pluripotency sustenance. Anim. Sci. J. 2019, 90, 1149-1160.
21. Kawaguchi, T.; Cho, D.; Hayashi, M.; Tsukiyama, T.; Kimura, K.; Matsuyama, S.; Minami, N.; Yamada, M.; Imai, H. Derivation of Induced Trophoblast Cell Lines in Cattle by Doxycycline-Inducible piggyBac Vectors. PLOS ONE 2016, 11, e0167550.
22. Canizo, J. R.; Vazquez Echegaray, C.; Klisch, D.; Aller, J. F.; Paz, D. A.; Alberio, R. H.; Alberio, R.; Guberman, A. S. Exogenous human OKSM factors maintain pluripotency gene expression of bovine and porcine iPS-like cells obtained with STEMCCA delivery system. BMC Res. Notes 2018, 11, 509.
23. Talbot, N. C.; Sparks, W. O.; Phillips, C. E.; Ealy, A. D.; Powell, A. M.; Caperna, T. J.; Garrett, W. M.; Donovan, D. M.; Blomberg, L. A. Bovine trophectoderm cells induced from bovine fibroblasts with induced pluripotent stem cell reprogramming factors. Mol. Reprod. Dev. 2017, 84, 468-485.
24. Su, Y.; Zhu, J.; Salman, S.; Tang, Y. Induced pluripotent stem cells from farm animals. J. Anim. Sci. 2020, 98, skaa343.
25. Zhao, L.; Gao, X.; Zheng, Y.; Wang, Z.; Zhao, G.; Ren, J.; Zhang, J.; Wu, J.; Wu, B.; Chen, Y.; et al. Establishment of bovine expanded potential stem cells. Proc. Natl. Acad. Sci. USA 2021, 118, 15.
26. Van Eenennaam, A. L. Application of genome editing in farm animals: Cattle. Transgenic Res. 2019, 28, 93-100.
27. Yuan, Y. Capturing bovine pluripotency. Proc. Natl. Acad. Sci. USA 2018, 115, 1962-1963.
28. Kehrli, M. E.; Ackermann, M. R.; Shuster, D. E.; Van Der Maaten, M. J.; Schmalstieg, F. C.; Anderson, D. C.; Hughes, B. J. Bovine leukocyte adhesion deficiency. Beta 2 integrin deficiency in young Holstein cattle. Am. J. Pathol. 1992, 140, 1489-1492.
29. Casal, M.; Haskins, M. Large animal models and gene therapy. Eur. J. Hum. Genet. 2006, 14, 266-272.
30. Wang, L.; Su, Y.; Huang, C.; Yin, Y.; Chu, A.; Knupp, A.; Tang, Y. NANOG and LIN28 dramatically improve human cell reprogramming by modulating LIN41 and canonical WNT activities. Biol. Open 2019, 8, 12.
31. Ludwig, T. E.; Levenstein, M. E.; Jones, J. M.; Berggren, W. T.; Mitchen, E. R.; Frane, J. L.; Crandall, L. J.; Daigh, C. A.; Conard, K. R.; Piekarczyk, M. S.; et al. Derivation of human embryonic stem cells in defined conditions. Nat. Biotechnol. 2006, 24, 185-187.
32. Tsukiyama, T.; Ohinata, Y. A Modified EpiSC Culture Condition Containing a GSK3 Inhibitor Can Support Germline-Competent Pluripotency in Mice. PLOS ONE 2014, 9, e95329. [PubMed] 33. Wu, J.; Luengo, A. P.; Sakurai, M.; Sugawara, A.; Gil, M. A.; Yamauchi, T.; Suzuki, K.; Bogliotti, Y. S.; Cuello, C.; Valencia, M. M.; et al. Interspecies Chimerism with Mammalian Pluripotent Stem Cells. Cell 2017, 168, 473-486.e15.
34. Matoba, S.; Liu, Y.; Lu, F.; Iwabuchi, K. A.; Shen, L.; Inoue, A.; Zhang, Y. Embryonic Development following Somatic Cell Nuclear Transfer Impeded by Persisting Histone Methylation. Cell 2014, 159, 884-895.
35. Chen, J.; Liu, H.; Liu, J.; Qi, J.; Wei, B.; Yang, J.; Liang, H.; Chen, Y.; Chen, J.; Wu, Y.; et al. H3K9 methylation is a barrier during somatic cell reprogramming into iPSCs. Nat. Genet. 2013, 45, 34-42.
36. Onder, T. T.; Kara, N.; Cherry, A.; Sinha, A. U.; Zhu, N.; Bernt, K. M.; Cahan, P.; Mancarci, B. O.; Unternaehrer, J.; Gupta, P. B.; et al. Chromatin-modifying enzymes as modulators of reprogramming. Nature 2012, 483, 598-602.
37. Soufi, A.; Donahue, G.; Zaret, K. S. Facilitators and Impediments of the Pluripotency Reprogramming Factors' Initial Engagement with the Genome. Cell 2012, 151, 994-1004.
38. Maherali, N.; Sridharan, R.; Xie, W.; Utikal, J.; Eminli, S.; Arnold, K.; Stadtfeld, M.; Yachechko, R.; Tchieu, J.; Jaenisch, R.; et al. Directly Reprogrammed Fibroblasts Show Global Epigenetic Remodeling and Widespread Tissue Contribution. Cell Stem Cell 2007, 1, 55-70.
39. Wernig, M.; Meissner, A.; Foreman, R.; Brambrink, T.; Ku, M.; Hochedlinger, K.; Bernstein, B. E.; Jaenisch, R. In vitro reprogramming of fibroblasts into a pluripotent ES-cell-like state. Nat. Cell Biol. 2007, 448, 318-324.
40. Thomson, J. A.; Itskovitz-Eldor, J.; Shapiro, S. S.; Waknitz, M. A.; Swiergiel, J. J.; Marshall, V. S.; Jones, J. M. Embryonic Stem Cell Lines Derived from Human Blastocysts. Science 1998, 282, 1145-1147.
41. Camus, A.; Perea-Gomez, A.; Moreau, A.; Collignon, J. Absence of Nodal signaling promotes precocious neural differentiation in the mouse embryo. Dev. Biol. 2006, 295, 743-755.
42. James, D.; Levine, A. J.; Besser, D.; Hemmati-Brivanlou, A. TGFbeta/activin/nodal signaling is necessary for the maintenance of pluripotency in human embryonic stem cells. Development 2005, 132, 1273-1282.
43. Vallier, L.; Reynolds, D.; Pedersen, R. A. Nodal inhibits differentiation of human embryonic stem cells along the neuroectodermal default pathway. Dev. Biol. 2004, 275, 403-421.
44. Ying, Q.-L.; Wray, J.; Nichols, J.; Batlle-Morera, L.; Doble, B.; Woodgett, J.; Cohen, P.; Smith, A. The ground state of embryonic stem cell self-renewal. Nat. Cell Biol. 2008, 453, 519-523.
45. Silva, J. C. R.; Barrandon, O.; Nichols, J.; Kawaguchi, J.; Theunissen, T.; Smith, A. Promotion of Reprogramming to Ground State Pluripotency by Signal Inhibition. PLOS Biol. 2008, 6, e253.
46. Smith, A.; Heath, J. K.; Donaldson, D. D.; Wong, G. G.; Moreau, J.; Stahl, M.; Rogers, D. Inhibition of pluripotential embryonic stem cell differentiation by purified polypeptides. Nat. Cell Biol. 1988, 336, 688-690.
47. Niwa, H.; Burdon, T.; Chambers, I.; Smith, A. Self-renewal of pluripotent embryonic stem cells is mediated via activation of STAT3. Genes Dev. 1998, 12, 2048-2060.
48. Lipsitz, Y. Y.; Woodford, C.; Yin, T.; Hanna, J. H.; Zandstra, P. W. Modulating cell state to enhance suspension expansion of human pluripotent stem cells. Proc. Natl. Acad. Sci. USA 2018, 115, 6369-6374. [PubMed]
49. Smith, A.; Guo, G.; Rostovskaya, M.; Clarke, J.; Dietmann, S.; Myers, S.; Bertone, P.; Reik, W. Epigenetic resetting of human pluripotency. Development 2017, 144, 2748-2763.
50. Hanna, J.; Cheng, A.; Saha, K.; Kim, J.; Lengner, C.; Soldner, F.; Cassady, J. P.; Muffat, J.; Carey, B. W.; Jaenisch, R. Human embryonic stem cells with biological and epigenetic characteristics similar to those of mouse ESCs. Proc. Natl. Acad. Sci. USA 2010, 107, 9222-9227.
51. Gafni, O.; Weinberger, L.; Mansour, A. A.; Manor, Y. S.; Chomsky, E.; Ben-Yosef, D.; Kalma, Y.; Viukov, S.; Maza, I.; Zviran, A.; et al. Derivation of novel human ground state naive pluripotent stem cells. Nature 2013, 504, 246-282.
52. Theunissen, T. W.; Powell, B. E.; Wang, H.; Mitalipova, M.; Faddah, D. A.; Reddy, J.; Fan, Z. P.; Maetzel, D.; Ganz, K.; Shi, L.; et al. Systematic identification of culture conditions for induction and maintenance of naive human pluripotency. Cell Stem Cell 2014, 15, 471-487.
53. Yeom, Y. I.; Fuhrmann, G.; Ovitt, C. E.; Brehm, A.; Ohbo, K.; Gross, M.; Hubner, K.; Scholer, H. R. Germline regulatory element of Oct-4 specific for the totipotent cycle of embryonal cells. Development 1996, 122, 881-894.
54. Tesar, P. J.; Chenoweth, J. G.; Brook, F. A.; Davies, T. J.; Evans, E. P.; Mack, D. L.; Gardner, R. L.; Mckay, R. D. G. New cell lines from mouse epiblast share defining features with human embryonic stem cells. Nat. Cell Biol. 2007, 448, 196-199.
55. Hanna, J.; Markoulaki, S.; Mitalipova, M.; Cheng, A. W.; Cassady, J. P.; Staerk, J.; Carey, B. W.; Lengner, C. J.; Foreman, R.; Love, J.; et al. Metastable pluripotent states in NOD-mouse-derived ESCs. Cell Stem Cell 2009, 4, 513-524.
56. Graf, A.; Krebs, S.; Zakhartchenko, V.; Schwalb, B.; Blum, H.; Wolf, E. Fine mapping of genome activation in bovine embryos by RNA sequencing. Proc. Natl. Acad. Sci. USA 2014, 111, 4139-4144.
57. Mallon, B. S.; Chenoweth, J. G.; Johnson, K. R.; Hamilton, R. S.; Tesar, P. J.; Yavatkar, A. S.; Tyson, L. J.; Park, K.; Chen, K.; Fann, Y. C.; et al. StemCellDB: The Human Pluripotent Stem Cell Database at the National Institutes of Health. Stem Cell Res. 2013, 10, 57-66.
58. Subramanian, A.; Tamayo, P.; Mootha, V. K.; Mukherjee, S.; Ebert, B. L.; Gillette, M. A.; Paulovich, A.; Pomeroy, S. L.; Golub, T. R.; Lander, E. S.; et al. Gene set enrichment analysis: A knowledge-based approach for interpreting genome-wide expression profiles. Proc. Natl. Acad. Sci. USA 2005, 102, 15545-15550.
59. Liberzon, A.; Subramanian, A.; Pinchback, R.; Thorvaldsdóttir, H.; Tamayo, P.; Mesirov, J. P. Molecular signatures database (MSigDB) 3.0. Bioinformatics 2011, 27, 1739-1740. [PubMed] 60. Liberzon, A.; Birger, C.; Thorvaldsdottir, H.; Ghandi, M.; Mesirov, J. P.; Tamayo, P. The Molecular Signatures Database (MSigDB) hallmark gene set collection. Cell Syst. 2015, 1, 417-425.
61. Krämer, A.; Green, J.; Pollard, J., Jr.; Tugendreich, S. Causal analysis approaches in Ingenuity Pathway Analysis. Bioinformatics 2014, 30, 523-530.
62. Takashima, Y.; Guo, G.; Loos, R.; Nichols, J.; Ficz, G.; Krueger, F.; Oxley, D.; Santos, F.; Clarke, J.; Mansfield, W.; et al. Resetting transcription factor control circuitry toward ground-state pluripotency in human. Cell 2018, 158, 1254-1269.
63. Brons, I. G. M.; Smithers, L. E.; Trotter, M.; Rugg-Gunn, P.; Sun, B.; Lopes, S. M. C. D. S.; Howlett, S. K.; Clarkson, A.; Ahrlund-Richter, L.; Pedersen, R. A.; et al. Derivation of pluripotent epiblast stem cells from mammalian embryos. Nat. Cell Biol. 2007, 448, 191-195.
64. Ghimire, S.; Van Der Jeught, M.; Neupane, J.; Roost, M. S.; Anckaert, J.; Popovic, M.; Van Nieuwerburgh, F.; Mestdagh, P.; Vandesompele, J.; Deforce, D.; et al. Comparative analysis of naive, primed and ground state pluripotency in mouse embryonic stem cells originating from the same genetic background. Sci. Rep. 2018, 8, 5884.
65. Rugg-Gunn, P.; Cox, B. J.; Lanner, F.; Sharma, P.; Ignatchenko, V.; McDonald, A. C.; Garner, J.; Gramolini, A. O.; Rossant, J.; Kislinger, T. Cell-Surface Proteomics Identifies Lineage-Specific Markers of Embryo-Derived Stem Cells. Dev. Cell 2012, 22, 887-901.
66. Xu, J.; Guo, Z.; Su, L.; Nedambale, T.; Zhang, J.; Schenk, J.; Moreno, J.; Dinnyes, A.; Ji, W.; Tian, X.; et al. Developmental Potential of Vitrified Holstein Cattle Embryos Fertilized In Vitro with Sex-Sorted Sperm. J. Dairy Sci. 2006, 89, 2510-2518.
67. Xiang, J.; Wang, H.; Zhang, Y.; Wang, J.; Liu, F.; Han, X.; Lu, Z.; Li, C.; Li, Z.; Gao, Y.; et al. LCDM medium supports the derivation of bovine extended pluripotent stem cells with embryonic and extraembryonic potency in bovine-mouse chimeras from iPSCs and bovine fetal fibroblasts. FEBS J. 2021, 288, 4394-4411.
68. Wang, L.; Huang, D.; Huang, C.; Yin, Y.; Vali, K.; Zhang, M.; Tang, Y. Enhanced human somatic cell reprogramming efficiency by fusion of the MYC transactivation domain and OCT4. Stem Cell Res. 2017, 25, 88-97.
69. Li, L.-C.; Dahiya, R. MethPrimer: Designing primers for methylation PCRs. Bioinformatics 2002, 18, 1427-1431.
70. Bock, C.; Reither, S.; Mikeska, T.; Paulsen, M.; Walter, J.; Lengauer, T. BiQ Analyzer: Visualization and quality control for DNA methylation data from bisulfite sequencing. Bioinformatics 2005, 21, 4067-4068.
71. Behringer, R.; Gertsenstein, M.; Nagy, K. V.; Nagy, A. Manipulating the Mouse Embryo: A Laboratory Manual, 4th ed.; Cold Spring Harbor Laboratory Press: Cold Spring Harbor, NY, USA, 2014.
72. Fan, Z.; Yang, M.; Regouski, M.; Polejaeva, I. A. Gene Knockouts in Goats Using CRISPR/Cas9 System and Somatic Cell Nuclear Transfer. Methods Mol. Biol. 2018, 1874, 373-390.
73. Kafi, M.; McGowan, M.; Kirkland, P. In vitro maturation and fertilization of bovine oocytes and in vitro culture of presumptive zygotes in the presence of bovine pestivirus. Anim. Reprod. Sci. 2002, 71, 169-179.

1. A cell culture medium comprising a WNT signaling inhibitor and a histone methyltransferase DOT1L inhibitor.

2. The cell culture medium of claim 1, wherein the WNT signaling inhibitor is selected from XAV-939, Wnt-C59, IWR1, ICG-001, IWP-2, LGK-974, CCT251545, FHF 535, IPW-4, JW 67, JW 74, KYA 1797K, NLS-StAx-H, TAK 715, WIC1, and derivatives thereof.

3. The cell culture medium of claim 2, wherein the WNT signaling inhibitor is IWR1.

4. The cell culture medium of claim 1, wherein the DOT1L inhibitor is selected from EPZ004777, EPZ-5676, and derivatives thereof.

5. The cell culture medium of claim 1, wherein the WNT signaling inhibitor is IWR1 and wherein the DOT1L inhibitor is EPZ004777.

6. The cell culture medium of claim 1, further comprising human leukemia inhibitory factor (LIF).

7. A cell culture medium comprising a WNT signaling inhibitor, a histone methyltransferase DOT1L inhibitor, human leukemia inhibitory factor (LIF), and an adenyl cyclase activator.

8. The cell culture medium of claim 7, wherein the WNT signaling inhibitor is selected from XAV-939, Wnt-C59, IWR1, ICG-001, IWP-2, LGK-974, CCT251545, FHF 535, IPW-4, JW 67, JW 74, KYA 1797K, NLS-StAx-H, TAK 715, WIC1, and derivatives thereof.

9. The cell culture medium of claim 7, wherein the DOT1L inhibitor is selected from EPZ004777, EPZ-5676, and derivatives thereof.

10. The cell culture medium of claim 7, wherein the adenyl cyclase activator is forskolin.

11. The cell culture medium of claim 7, wherein the WNT signaling inhibitor is IWR1, the DOT1L inhibitor is EPZ004777, and the adenyl cyclase activator is forskolin.

12. The cell culture medium of claim 7, further comprising at least one glycogen synthase kinase 3 (GSK-3) inhibitor and at least one MEK1/2 inhibitor.

13. The cell culture medium of claim 12, wherein the GSK-3 inhibitor is selected from CHIR-99021, CHIR-98014, LY2090314, BIO, TWS119, Tideglusib, SB216763, and derivatives thereof.

14. The cell culture medium of claim 12, wherein the GSK-3 inhibitor is CHIR-99021.

15. The cell culture medium or claim 12, wherein the MEK1/2 inhibitor is selected from Trametinib, PD0325901, Selumetinib, UO126-EtOH, PD989059, and derivatives thereof.

16. The cell culture medium or claim 15, wherein the MEK1/2 inhibitor is PD0325901.

17. The cell culture medium of claim 12, wherein the GSK-3 inhibitor is CHIR-99021 and the MEK1/2 inhibitor is PD0325901.

18. A method of producing a bovine-induced pluripotent stem cell, the method comprising:
a. providing a bovine mesenchymal stem cell, and
b. inducing overexpression of a plurality of reprogramming factors in the bovine mesenchymal stem cell, wherein the plurality of reprogramming factors comprise lysine-specific demethylase 4A (KDM4A), OCT4, SOX2, KLF4, cMYC, LIN28, and NANOG.

19. The method of claim 18, further comprising culturing the mesenchymal stem cells under suitable conditions to promote cell reprogramming.

20. The method of claim 19, wherein culturing the cell under suitable conditions to promote cell reprogramming comprises culturing the cell in the cell culture medium of any one of the preceding claims.

21. The method of claim 20, wherein culturing the cell under suitable conditions to promote cell reprogramming comprises culturing the cell in the medium of claim 1.

22. A bovine-induced pluripotent stem cell produced by the method of claim 18.


SEQUENCE LISTING (12 sequences)

SEQ ID NO: 1 - DNA, 30 bp, synthetic construct
  source 1..30: mol_type = other DNA
gtttggagaggggttttgaagaatgtgtag

SEQ ID NO: 2 - DNA, 28 bp, synthetic construct
  source 1..28: mol_type = other DNA
atcccacccactaaccttaacctctaac

SEQ ID NO: 3 - DNA, 25 bp, synthetic construct
  source 1..25: mol_type = other DNA
tgtttggagaattttatggatagag

SEQ ID NO: 4 - DNA, 25 bp, synthetic construct
  source 1..25: mol_type = other DNA
ccaattaaatcatcaaacctaactc

SEQ ID NO: 5 - DNA, 30 bp, synthetic construct
  source 1..30: mol_type = other DNA
taggtggttataggagatgtatttttgatt

SEQ ID NO: 6 - DNA, 30 bp, synthetic construct
  source 1..30: mol_type = other DNA
ttataaataaaactcaaccatacttaaccc

SEQ ID NO: 7 - DNA, 20 bp, synthetic construct
  source 1..20: mol_type = other DNA
tgtgggcaaagaggcttcat

SEQ ID NO: 8 - DNA, 20 bp, synthetic construct
  source 1..20: mol_type = other DNA
caaagctgacttagcctcag

SEQ ID NO: 9 - DNA, 20 bp, synthetic construct
  source 1..20: mol_type = other DNA
tgaggcatggaactccgctt

SEQ ID NO: 10 - DNA, 23 bp, synthetic construct
  source 1..23: mol_type = other DNA
ggtggttccacattccgtaggac

SEQ ID NO: 11 - DNA, 20 bp, synthetic construct
  source 1..20: mol_type = other DNA
ccggtcgctaccattaccag

SEQ ID NO: 12 - DNA, 20 bp, synthetic construct
  source 1..20: mol_type = other DNA
cggccgctcgagtttaaata